(19) United States
(12) Patent Application Publication
(10) Pub. No.: US 2025/0177551 A1
(43) Pub. Date: Jun. 5, 2025

(54) ENGINEERED RED BLOOD CELLS TARGETING PD-1

(71) Applicants: WESTLAKE THERAPEUTICS (SHANGHAI) CO., LIMITED, Shanghai (CN); WESTLAKE LABORATORY OF LIFE SCIENCES AND BIOMEDICINE, Zhejiang (CN)

(72) Inventors: Xiaofei GAO, Zhejiang (CN); Yanjie HUANG, Shanghai (CN); Xiaoqian NIE, Shanghai (CN); Xuan LIU, Shanghai (CN); Yang YUAN, Shanghai (CN)

(21) Appl. No.: 18/845,846

(22) PCT Filed: Mar. 9, 2023
(86) PCT No.: PCT/CN2023/080590

§ 371 Date: Sep. 10, 2024

(30) Foreign Application Priority Data
Mar. 11, 2022 (CN) 202210238234.4

(51) Int. Cl. A61K 47/68; A61K 47/69; A61P 35/00

(52) U.S. Cl. CPC A61K 47/6849; A61K 47/6901; A61P 35/00

(57) ABSTRACT

The present invention relates to a type of red blood cell targeting PD-1, specifically an engineered red blood cells loading anti-PD-1 antibodies, preparation methods thereof, and methods and uses of the same in treating cancers, particularly cancers that are insensitive or resistant to anti-PD-1 antibodies.

The present invention relates to a type of red blood cell targeting PD-1, specifically an engineered red blood cells loading anti-PD-1 antibodies, preparation methods thereof, and methods and uses of the same in treating cancers, particularly cancers that are insensitive or resistant to anti-PD-1 antibodies.

### BACKGROUND

Metabolisms of erythrocyte (red blood cell, RBC) are completed generally via an in-vivo clearance in the spleen that acts as a filter for the blood circulation, as indicated in numerous studies (Structure and function of the immune system in the spleen. Nat Rev Immunol. 2005 August; 5 (8): 606-16; The spleen in local and systemic regulation of immunity. Immunity. 2013 Nov. 14; 39 (5): 806-18). Consequently, erythrocytes are considered as an excellent transporter for targeting the spleen, enabling the targeted delivery of immunotherapeutic drugs. A research team from Harvard University has successfully induced tumor regression in a mouse tumor model by using erythrocyte-driven immunization, loading the OVA antigen as a therapeutic vaccine to activate specific T-cells (Erythrocyte-driven immunization via biomimicry of their natural antigen-presenting function. Samir Mitragotri et al. Proc Natl Acad Sci USA. 2020 Jul. 28; 117 (30): 17727-17736). Research suggests that carrying drugs on cells typically achieve better drug-cell interactions (Membrane interactions in drug delivery: Model cell membranes and orthogonal techniques. Adv Colloid Interface Sci. 2020 July; 281:102177; Drug delivery by erythrocytes: “Primum non nocere”. Transfus Apher Sci. 2016 December; 55 (3): 275-280.). In recent years, some products that employ erythrocytes for drug delivery have already been subjected to clinical research, wherein the mechanism for some of these products involves entering the spleen to activate immune cells (Anti-tumor effects of RTX-240: an engineered red blood cell expressing 4-1BB ligand and interleukin-15. Sivan Elloul et al. Cancer Immunol Immunother. 2021 September; 70 (9): 2701-2719.).

Programmed cell death protein 1, also known as PD-1 or CD279, is a cell surface protein, which downregulates the response of immune system to human cells by binding to its ligand PD-L1. PD-1 regulates the immune system and promotes self-tolerance by inhibiting the inflammatory activity of T cells, which can prevent autoimmune diseases, but also prevents the immune system from killing cancer cells. PD-1 is an immune checkpoint that prevent autoimmunity via two mechanisms as follows: firstly, PD-1 promotes the apoptosis (programmed cell death) of antigen-specific T cells in the lymph nodes; secondly, PD-1 reduces the apoptosis of regulatory T cells (anti-inflammatory, suppressor T cells). In neoplastic disease state, PD-L1 on tumor cells and PD-1 on T cells interact with each other to reduce the function signal of T cells, thus preventing the immune system to attack tumor cells. The use of inhibitors (monoclonal antibodies) for blocking the interaction between PD-L1 and PD-1 receptors can reactivate some dysfunctional cells and also prevent T cells provided by the peripheral immune system from becoming tolerant T cells, thus achieving cancer therapy. The use of anti-PD-1 monoclonal antibodies may achieve significant therapeutic effects clinically directed to cancers including melanoma, head-neck carcinoma, renal cell carcinoma, non-small cell lung cancer, bladder cancer, and colorectal cancer, etc. However, a subset of patients with these indications still cannot benefit from PD-1 antibody therapy mainly because PD-1 antibodies primarily act on T cells in tumor tissues, wherein the T cells will occur severe dysfunction at advanced stages of tumors. It is difficult to reactivate those T cells by blocking the PD-1-PD-L1 pathway, exhibiting resistance to PD-1 antibodies (A Burned-Out CD8+ T-cell Subset Expands in the Tumor Microenvironment and Curbs Cancer Immunotherapy. Cancer Discov. 2021 July; 11 (7): 1700-1715. doi: 10.1158/2159-8290.CD-20-0962. Epub 2021 Mar. 3. PMID: 33658301.) (Peripheral T cell expansion predicts tumour infiltration and clinical response. Nature. 2020 March; 579 (7798): 274-278. doi: 10.1038/s41586-020-2056-8. Epub 2020 Feb. 26. PMID: 32103181.).

Therefore, there is still a need for more effective products for cancer treatments, particularly products that can activate the peripheral immune system preferably and/or more effectively treat cancers (such as cancers that cannot benefit from PD-1 inhibitor therapy).

### SUMMARY

The present invention, therefore, provides red blood cells loaded with PD-1 inhibitors, specifically red blood cells loaded with anti-PD-1 antibodies (red blood cells-PD-1 antibody conjugate, or RBC-Anti-PD-1, or Anti-PD-1-RBC), which exhibit one or more of the following properties, particularly one or more of the following improved properties compared to the corresponding PD-1 inhibitors not loaded on red blood cells:
1) Enhancing the therapeutic efficacy of the conjugated PD-1 inhibitor, for instance, in cancers, such as lung cancer, pancreatic cancer, breast cancer, and gastrointestinal cancers including colon cancer, colorectal cancer, or rectal cancer, etc., exhibiting superior tumor-suppressive effects and/or requiring a lower effective dose; specifically, for example
   (a) exhibiting superior tumor-suppressive effects in PD-1 inhibitor-sensitive colorectal cancers, for example, in an in vivo PD-1 inhibitor-sensitive mouse MC38 tumor model;
   (b) requiring a lower effective dose in PD-1 inhibitor-sensitive colorectal cancers, for example, in an in vivo PD-1 inhibitor-sensitive mouse MC38 tumor model;
2) Showing effectiveness against cancers that are resistant or insensitive to PD-1 inhibitors; for instance, exhibiting superior tumor-suppressive effects and/or requiring a lower effective dose in lung cancer, pancreatic cancer, breast cancer, and gastrointestinal cancers including colon cancer, colorectal cancer, or rectal cancer, etc., which are insensitive to PD-1 inhibitor therapy; specifically, for example
   (a) exhibiting superior tumor-suppressive effects in PD-1 inhibitor-insensitive lung cancers, for example, in an in vivo PD-1 inhibitor-insensitive mouse lung cancer KP model;
   (b) exhibiting superior tumor-suppressive effects in PD-1 inhibitor-insensitive breast cancers, for example, in an in vivo PD-1 inhibitor-insensitive mouse breast cancer PB3 model;
3) Exhibiting an enhanced PD-1-PD-L1 binding blocking capability, for instance, exhibiting an enhanced PD-1-PD-L1 binding blocking capability compared to the PD-1 inhibitors at equivalent dosages in the cellular activity assays in vitro;
4) Improving the in vivo distribution (for example, in mice or humans) of PD-1 inhibitors, for instance, enriching a higher proportion of PD-1 inhibitors in the spleen and/or activating more effector CD8⁺ T cells within the spleen;
5) Activating a higher proportion of CD8⁺ T cells compared to the corresponding PD-1 inhibitors at equivalent dosages;
6) Activating the immune system and immune cells more effectively compared to the corresponding PD-1 inhibitors at equivalent dosages, for instance, activating a greater number of immunotherapeutic markers, for example, activating Luciferous fluorescence signal (RFU) in the downstream pathway of NFAT Reporter more significantly compared to the corresponding PD-1 inhibitors that are not loaded on red blood cells even at a lower dose, indicating they may activate the immune system and immune cells more effectively;
7) Exhibiting a certain level of safety in acute toxicity experiments, for instance, exhibiting a certain level of safety in mouse acute toxicity experiments.

The present invention further provides a method for engineering mature natural red blood cells to enable their efficient loading with therapeutic agents. The red blood cell engineering method herein offers one or more of the following advantages over the engineering modification methods of red blood cells derived from stem cells, such as enhanced ease of material acquisition, a simplified process workflow, and a reduced time frame from the collection of peripheral blood to the acquirement of engineered red blood cells, for example, within as little as 2 days.

The present invention further provides a method for the covalent attachment of therapeutic agents to red blood cells, such as covalent attachment to the membrane proteins of the red blood cells, thereby yielding engineered red blood cells loaded with therapeutic agents. The method is less damaging to erythrocytes compared to established hypotonic methods, for instance, it does not compromise the physiological integrity of the erythrocytes. In some embodiments, the therapeutic agent is an antibody. In some embodiments, the therapeutic agent is a PD-1 inhibitor, such as a PD-1 antibody.

The present invention further provides a method employing red blood cells loaded with PD-1 inhibitors, as described herein, for the treatment of diseases, such as tumors, and such as cancers.

The present invention further provides a use of red blood cells loaded with PD-1 inhibitors for the treatment of diseases, such as tumors, and such as cancers.

The present invention further provides a use of red blood cells loaded with PD-1 inhibitors in the preparation of medicament for the treatment of diseases, such as tumors, and such as cancers.

### BRIEF DESCRIPTION OF THE DRAWINGS

FIG. 1A shows the results of the immunohistochemical staining experiments for RBC-Keytruda. FIG. 1B and FIG. 1C show the results of the PD-1/PD-L1 blocking bioassays, wherein FIG. 1B shows a comparison of the in vitro activity of RBC-Keytruda and Keytruda at the same low dosage; FIG. 1C shows a comparison of the in vitro activity of RBC-Keytruda and Keytruda at the same Keytruda dosage.

FIG. 2 shows the change curve of tumors in mice.

FIG. 3 shows the survival rate curve of mice.

FIG. 4 shows the change curve of tumors in mice.

FIG. 5 shows the change in the total number of tumor-infiltrating CD8⁺ cells in mice.

FIG. 6 shows the change curve of tumors in mice.

FIG. 7 shows the comparison of tumor weight at the endpoint.

FIG. 8 shows the proportion of Cell Trace Far Red positive cells in the peripheral blood of mice.

FIG. 9 shows the proportion of Keytruda positive cells among the Cell Trace Far Red positive cells.

FIG. 10 shows an exemplary method for constructing antibody-erythrocyte conjugates.

FIG. 11 shows the results of the conjugation efficiency assays for the antibody-erythrocyte conjugates.

FIG. 12 shows the results of in vitro activity assays for the antibody-erythrocyte conjugates.

FIG. 13 shows the results of in vivo pharmacokinetic assays for the antibody-erythrocyte conjugates.

### DETAILED DESCRIPTION

### I. Definition

Before proceeding with a detailed description of the present invention, it should be understood that the invention is not limited to the specific methodologies, schemes, and reagents described herein, as these may vary. It should also be understood that the terms used herein are intended solely for the purpose of describing specific embodiments and are not intended to limit the scope of the invention, which will be limited only by the claims appended hereto. Unless otherwise defined, all technical and scientific terms used herein have the same meaning as generally understood by those of ordinary skilled in the art to which the invention pertains.

For the purpose of this specification, the following definitions will be used, and terms used in the singular may also be construed to include the plural, and vice versa, where appropriate. It is to be understood that the terms used herein are intended solely to describe specific embodiments and are not intended to be limiting.

The term “about” when used in conjunction with numerical values means to encompass numerical values within a range that includes the lower limit which is 5% less than the specified numerical value and the upper limit which is 5% more than the specified numerical value.

As used herein, the term “and/or” means any one of the options, or both or multiple of the options.

As used herein, the terms “comprising” or “including” are intended to mean that the elements, integers, or steps mentioned are included, but do not exclude any other elements, integers, or steps. When the terms “comprising” or “including” are used herein, unless otherwise specified, they also encompass situations that are composed of the mentioned elements, integers, or steps. For example, when it is stated that “comprising” an antibody variable region of a specific sequence, it is also intended to encompass the variable region of the antibody composed of that specific sequence.

The terms “anti-(human) PD-1 antibody”, “anti-(human) PD-1”, “(human) PD-1 antibody”, “antibody binding to (human) PD-1”, or “antibody specifically binding to (human) PD-1” as used herein refer to antibodies that can bind to (human) PD-1 with sufficient affinity so that the said antibodies may be used as therapeutic agents targeting (human) PD-1. In one embodiment, the described (human) PD-1 antibody binds to (human) PD-1 with high affinity in vitro or in vivo. There are various anti-PD-1 antibodies known in the field that can be used for treatment, such as commercially available antibodies, for example, pembrolizumab from Merck, camrelizumab from Hengrui, tislelizumab from BeiGene, sintilimab from Innovent, toripalimab from Junshi Biosci, zimberelimab from Gloria Biosci, penpulimab from Chiatai tianqing, pucotenlimab from Lepu Biopharma, serplulimab from Henlius Biotech, or nivolumab from BMS.

The terms “complete antibody”, “whole antibody” or “full-length antibody” as used herein are interchangeable and refer to antibody molecules with the structures of natural immunoglobulin molecules. In the case of conventional four-chain IgG antibodies, full-length antibodies consist of two heavy chains (H) and two light chains (L) connected by disulfide bonds. In the case of heavy-chain-only antibodies that have only heavy chains and lack light chains, full-length antibodies consist of two heavy chains (H) connected by disulfide bonds.

The term “antibody fragment” refers to a part of a complete antibody. In a preferred embodiment, the antibody fragment is an antigen-binding fragment.

The “antigen-binding fragment” refers to a molecule that is different from the complete antibody, which includes a part of the complete antibody and binds to the antigen that the complete antibody binds to. Examples of antibody fragments include but are not limited to a Fv, a Fab, a Fab′, a Fab′-SH, a F(ab′)₂, a dAb (domain antibody), a linear antibody, a single-chain antibody (e.g., scFv), a single-domain antibody such as VHH, a bispecific antibody or fragment thereof, or a camelid antibody.

The term “antigen” refers to a molecule that elicits an immune response. This immune response can involve the production of antibodies, or the activation of specific immune cells, or both. It is understood by those skilled in the art that any macromolecule, including essentially all proteins or peptides, can serve as an antigen. Furthermore, antigens can be derived from recombinant or genomic DNA. In some embodiments, the antigen is PD-1, such as human PD-1. As used herein, the term “epitope” refers to the part of the antigen (e.g., PD-1) that interacts specifically with the antibody molecule.

The “Complementarity Determining Region” or “CDR region” or “CDR” refers to the region within the variable domains of an antibody that is highly variable in sequence and forms structurally defined loops (“hypervariable loops”) and/or contains antigen-contacting residues (“antigen-contacting sites”). The CDR is primarily responsible for binding to the epitope of an antigen. The CDRs of the heavy chains and light chains are commonly referred to as CDR1, CDR2, and CDR3, numbered sequentially from the N-terminus. The CDRs located within the variable domain of the antibody heavy chain are referred to as HCDR1, HCDR2, and HCDR3, while those within the variable domain of the antibody light chain are referred to as LCDR1, LCDR2, and LCDR3. The precise amino acid sequence boundaries of each CDR in a given amino acid sequence of the variable region of a light chain or the variable region of a heavy chain can be determined using any or a combination of several well-known antibody CDR assignment systems, including: the Chothia system based on the three-dimensional structure of antibody and topology of CDR loops (Chothia et al. (1989) Nature 342:877-883, Al-Lazikani et al., “Standard conformations for the canonical structures of immunoglobulins”, Journal of Molecular Biology, 273, 927-948 (1997)); the Kabat system based on the variability of antibody sequences (Kabat et al., Sequences of Proteins of Immunological Interest, 4th Edition, U.S. Department of Health and Human Services, National Institutes of Health (1987)), AbM (University of Bath), Contact (University College London), the international ImMunoGeneTics database (IMGT) (available on the world wide web at imgt.cines.fr/); and the North CDR definition based on affinity propagation clustering using a large number of crystal structures (North et al., “A New Clustering of Antibody CDR Loop Conformations”, Journal of Molecular Biology, 406, 228-256 (2011)).

There are CDR region ranges defined using the Kabat, AbM, Chothia, Contact, and IMGT schemes below.

| | Kabat | AbM | Chothia | Contact | IMGT |
|---|---|---|---|---|---|
| CDR | scheme | scheme | scheme | scheme | scheme |
| LCDR1 (Kabat and | L24-L34 | L24-L34 | L26-L32 | L30-L36 | L27-L32 |
| Chothia numbering | | | | | |
| systems) | | | | | |
| LCDR2 (Kabat and | L50-L56 | L50-L56 | L50-L52 | L46-L55 | L50-L52 |
| Chothia numbering | | | | | |
| systems) | | | | | |
| LCDR3 (Kabat and | L89-L97 | L89-L97 | L91-L96 | L89-L96 | L89-L96 |
| Chothia numbering | | | | | |
| systems) | | | | | |
| HCDR1 (Kabat | H31-H35B | H26-H35B | H26-H32 | H30-H35B | H26-H35B |
| numbering system) | | | | | |
| HCDR1 (Chothia | H31-H35 | H26-H35 | H26-H32 | H30-H35 | H26-H35 |
| numbering system) | | | | | |
| HCDR2 (Kabat and | H50-H65 | H50-H58 | H53-H55 | H47-H58 | H51-H57 |
| Chothia numbering | | | | | |
| systems) | | | | | |
| HCDR3 (Kabat and | H95-H102 | H95-H102 | H96-H101 | H93-H101 | H93-H102 |
| Chothia numbering | | | | | |
| systems) | | | | | |

CDR can also be determined based on having the same Kabat numbering position as a reference CDR sequence (for example, any of the exemplary CDRs as described herein).

The term “CDR” or “CDR sequence” encompasses CDR sequences determined in any of the aforementioned manners, unless otherwise specified herein.

Unless otherwise specified herein, when referring to the positions of residues in the variable regions of antibodies (including both heavy chain variable region residues and light chain variable region residues), it is in reference to the numbering positions of the “Kabat numbering system” described by Kabat et al., Sequences of Proteins of Immunological Interest, 5th Edition, Public Health Service, National Institutes of Health, Bethesda, Md. (1991).

The term “Fc region” is used herein to define the C-terminal region of the immunoglobulin heavy chain that contains at least a part of the constant region. This term includes both the native sequence Fc region and variant Fc region. The natural immunoglobulin “Fc domain” consists of two or three constant domains, namely the CH2 domain, the CH3 domain, and the optional CH4 domain. For example, in natural antibodies, the immunoglobulin Fc domain includes the second and third constant domains (CH2 domain and CH3 domain) derived from two heavy chains of IgG, IgA, and IgD class antibodies; or it includes the second, third, and fourth constant domains (CH2 domain, CH3 domain, and CH4 domain) derived from two heavy chains of IgM and IgE class antibodies. Unless otherwise specified herein, the amino acid residue numbering in the Fc region or the heavy chain constant region is numbered according to the EU numbering system (also known as the EU index) as described by Kabat et al., Sequences of Proteins of Immunological Interest, 5th Edition, Public Health Service, National Institutes of Health, Bethesda, MD, 1991. The term “Fc region” as described herein does not include the variable region of a heavy chain VH and the variable region of a light chain VL, nor the constant region of heavy chain CH1 and the constant region of light chain CL of immunoglobulin, but may include the hinge region at the N-terminus of the constant region of heavy chain in some cases.

The “antibody in the form of IgG” refers to an antibody whose constant region of heavy chain belongs to the IgG class. For example, an antibody in the form of IgG4 indicates that its constant region of heavy chain is derived from IgG4, or an antibody in the form of IgG1 indicates that its constant region of heavy chain is derived from IgG1.

The “erythrocyte” or “RBC” or the “red blood cell” is the most common type of blood cells and also serve as the primary carrier for the transport of oxygen from the circulatory system to the body's tissues through bloodstreams in vertebrates. The cytoplasm of erythrocytes is rich in hemoglobin, an iron-containing biomolecule that can bind to oxygen, giving the cells and blood their red color. The cell membrane of erythrocytes is composed of proteins and lipids, and this kind of structure provides the necessary characteristics for physiological cellular functions, such as deformability and stability, allowing it to traverse the circulatory system, especially the capillary network. The mature human erythrocytes are flexible, oval biconcave discs, which lack a nucleus and most organelles to maximize the space for hemoglobin; and can be considered as bags of hemoglobin enclosed by the plasma membranes. Approximately 2.4 million new erythrocytes are produced per second in adults. In the human body, approximately 84% of the cells (20-30 trillion) are erythrocytes and nearly half of the blood volume (between 40% and 45%) consists of erythrocytes.

The “mature natural red blood cells” or “mature natural erythrocytes” or “natural mature blood cells” or “natural mature erythrocytes” refer to mature natural red blood cells that are directly isolated and obtained from the blood of animals, particularly humans (such as adults or children).

The “blood preparation” or “blood product” as described herein is interchangeable and refers to products prepared from (human) blood for medical use. Blood preparations include whole blood preparations, component blood preparations, plasma preparations, or leukoreduced blood preparations.

The term “therapeutic agent” as described herein encompasses any substance that is effective in the prevention or treatment of tumors, such as cancer, including chemotherapeutic agents, cytotoxic agents, other antibodies, vaccines, small molecule drugs, or immunomodulators (such as immunosuppressants or immune agonists).

The term “cytotoxic agent” as used herein refers to agents that inhibit or prevent cellular function and/or cause cell death or destruction.

The “chemotherapeutic agent” includes useful chemical compounds in the treatment of cancer or immune system diseases.

The term “small molecule drug” refers to compounds with low molecular weight that are capable of modulating biological processes. “Small molecules” are defined as molecules with a molecular weight less than 10 kD, typically less than 2 kD, and preferably less than 1 kD. Small molecules include but are not limited to, inorganic molecules, organic molecules, organic molecules containing inorganic components, molecules containing radioactive atoms, synthetic molecules, peptidomimetics, and antibody mimetics. As therapeutic agents, small molecules can penetrate cells more readily than large molecules, with less susceptible to degradation, and less likely to provoke an immune response.

The term “immunomodulator” as used herein refers to natural or synthetic active agents or drugs that suppress or regulate the immune response. The immune response can be either a humoral response or a cellular response. Immunomodulators include immunosuppressants or immune agonists. In some embodiments, the immunomodulators as described herein include immune checkpoint inhibitors or immune checkpoint agonists.

The term “effective amount” refers to such an amount or dose of the antibodies or fragments or compositions or preparations or products or combinations or combination products as described herein, when administered to a patient in a single dose or multiple doses, to produce the desired effect in patients requiring treatment or prevention.

The “therapeutically effective amount” refers to the amount that, when administered at the required dosage and for the necessary duration, effectively achieves the desired therapeutic outcome. A therapeutically effective amount is also such an amount where any toxic or harmful effects of the antibodies or antibody fragments or compositions or preparations or products or combinations or combination products are not greater than the beneficial therapeutic effects. Preferably, the “therapeutically effective amount” inhibits a measurable parameter (such as tumor volume) by at least about 30%, and even more preferably by at least about 40%, 45%, 50%, 55%, 60%, 65%, 70%, 75%, 80%, 85%, 90%, or even 100% relative to an untreated subject.

The “prophylactically effective amount” refers to the amount that, when administered at the required dosage and for the necessary duration, effectively achieves the desired prophylactic outcome. Typically, because prophylactic doses are used before or at an early stage of the disease in the subject, the prophylactically effective amount will be less than the therapeutically effective amount.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cells and their progeny, regardless of the number of passages. The progeny may not be identical to the parent cells in terms of nucleic acid content and may contain mutations. The mutant progenies screened or selected for the same functionality or biological activity from the initially transformed cells are included herein.

The term “label” as used herein refers to a compound or composition that is either directly or indirectly conjugated or fused to a reagent (such as a polynucleotide probe or an antibody) and facilitates the detection of the conjugated or fused reagent. The label itself can be detectable (for example, a radioactive isotope label or a fluorescent label) or, in the case of enzymatic labeling, can catalyze a chemical change in a detectable substrate compound or composition. The term is intended to encompass both direct labeling of the probe or antibody by coupling (i.e., physically linking) a detectable substance to the probe or antibody and indirect labeling of the probe or antibody by reacting with another reagent that is directly labeled.

The “individual” or “subject” includes the mammal. Mammals include but are not limited to, domestic animals (e.g., cattle, sheep, cats, dogs, and horses), primates (e.g., humans and non-human primates like monkeys), rabbits, and rodents (e.g., mice and rats). In some embodiments, the individual or subject is a human.

The “isolated” antibody or other molecule (such as an ADC molecule or a nucleic acid molecule) refers to an antibody or molecule that has been separated from its natural environment or the components of the environment in which it is expressed. In some embodiments, the antibody or ADC molecule is purified to a purity of over 95% or 99%, as determined by methods such as electrophoresis (e.g., SDS-PAGE, isoelectric focusing (IEF), capillary electrophoresis) or chromatography (e.g., ion exchange or reverse-phase HPLC).

The term “anti-tumor activity” refers to the biological effect that can be demonstrated by various means, including but not limited to, for example, reduction in tumor volume, reduction in the number of tumor cells, reduction in tumor cell proliferation, or reduction in tumor cell survival.

The terms “tumor” and “cancer” are used herein interchangeably to encompass both solid tumors and hematological tumors.

The term “cancer” refers to or describes a physiological disorder in mammals, characterized typically by unregulated cell growth. In some embodiments, cancers suitable for treatment with the antibodies as described herein include lung cancer, pancreatic cancer, breast cancer, gastrointestinal tumors such as colon cancer, colorectal cancer, or rectal cancer, including their metastatic forms.

The term “tumor” refers to all neoplastic cell growth and proliferation, whether malignant or benign, as well as all pre-cancerous and cancerous cells and tissues. The terms “cancer” and “tumor” are not mutually exclusive when mentioned herein.

The term “pharmaceutical excipient” refers to diluents, adjuvants (such as Freund's adjuvant (complete and incomplete)), excipients, carriers, or stabilizers, etc., which are administered together with the active ingredient.

The term “pharmaceutical composition” refers to a composition that allows the active ingredient contained therein to exist in a biologically active form and does not contain additional components that have unacceptable toxicity to the subject to which the composition is administered.

The term “pharmaceutical combination” refers to either a non-fixed combination product or a fixed combination product, including but not limited to pharmaceutical kits and pharmaceutical combinations. The term “non-fixed combination” means that the active ingredients (e.g., (i) the antibody-erythrocyte conjugates as described herein, and (ii) other therapeutic agents) are administered to the patient as separate entities simultaneously, without specific time constraints or at the same or different intervals, in a sequential manner, wherein such administrations provide the patient with a preventive or therapeutically effective level of two or more active ingredients. In some embodiments, the antibody-erythrocyte conjugates as described herein and other therapeutic agents used in the pharmaceutical combination are administered at a level not exceeding the levels at which they are used individually. The term “fixed combination” means that two or more active ingredients are simultaneously administered to the patient in the form of a single entity. Preferably, the dosage and/or timing interval of the two or more active ingredients is selected so that the combined use of the parts can produce an effect greater than that achievable by using any one component alone in the treatment of diseases or conditions. Each component can be presented in a separate preparation, and the preparation can be the same or different.

The term “combination therapy” refers to the administration of two or more therapeutic agents or treatment modalities (such as radiation therapy or surgery) to treat the diseases described herein. This administration includes the co-administration of these therapeutic agents in a substantially simultaneous manner, for example, in a single capsule with a fixed ratio of active ingredients. Alternatively, this administration includes the co-administration of each active ingredient in multiple or separate containers (such as tablets, capsules, powders, and liquids). The powders and/or liquids can be reconstituted or diluted to the required dosage before administration. Furthermore, this administration also includes the use of each type of therapeutic agent or treatment modality at approximately the same time or at different times in a sequential manner. In either case, the therapeutic regimen will provide the beneficial effects of combination therapy in treating the diseases or conditions described herein.

As used herein, “treatment” refers to the alleviation, interruption, obstruction, mitigation, cessation, reduction, or reversal of the progression or severity of existing symptoms, illnesses, conditions, or diseases.

As used herein, “prevention” includes the inhibition of the occurrence or development of diseases or illnesses, or symptoms of specific diseases or illnesses. In some embodiments, subjects with a family history of cancer are candidates for a preventive regimen. Typically, in the context of cancer, the term “prevention” refers to the administration of medication before the onset of cancer symptoms or signs, especially in subjects at risk of cancer.

The term “vector” as used herein refers to a nucleic acid molecule capable of replicating another nucleic acid to which it is linked. This term includes vectors that are capable of self-replication of nucleic acid structures, as well as vectors that integrate into the genome of the host cell into which they have been introduced. Some vectors are capable of directing the expression of the nucleic acid with which they are operably linked. Such vectors are referred to as “expression vectors” herein.

“Subject/patient/individual samples” refer to the collection of cells or fluids obtained from patients or subjects. The source of tissue or cell samples can be solid tissues, such as from fresh, frozen, and/or preserved organ or tissue samples or biopsy samples or puncture samples; blood or any blood component; body fluids, such as cerebrospinal fluid, amniotic fluid, peritoneal fluid (ascites), or interstitial fluid; and cells at any time of pregnancy or development from the subject. Tissue samples may contain compounds that are naturally not mixed with tissues in nature, such as preservatives, anticoagulants, buffers, fixatives, nutrients, antibiotics, etc.

### II. Antibody-Erythrocyte Conjugate

The present disclosure relates to an antibody-erythrocyte conjugate, which comprises an erythrocyte conjugated with an antibody or antigen-binding fragment thereof. Preferably, the antibody or antigen-binding fragment thereof is conjugated to the erythrocytes via a linker. Preferably, the antibody or antigen-binding fragment thereof is conjugated to a membrane protein on the erythrocytes, for example, the antibody or antigen-binding fragment thereof is conjugated to the membrane protein on the erythrocytes via a linker.

In some embodiments, the erythrocytes are mature natural erythrocytes, preferably from human mature natural erythrocytes.

In one embodiment, the antibody can be connected with 1, 2, 3, 4, 5, 6, 7, 8, 9, or 10 linkers. In one embodiment, the number of antibodies on a single erythrocyte can be between 10³ and 10⁶, for example, between approximately 10⁴ and 10⁶ or between 10⁵ and 10⁶, for instance, about 1×10⁵, 2×10⁵, 3×10⁵, 4×10⁵, 5×10⁵, 6×10⁵, 7×10⁵, 8×10⁵, or 9×10⁵.

In some embodiments, the antibody or antigen-binding fragment thereof herein is an antibody or antigen-binding fragment thereof that binds to PD-1, particularly the human PD-1.

Any PD-1 antibody or antigen-binding fragment thereof known to those skilled in the art is applicable to the present invention. The antibodies described include but are not limited to, those that have been disclosed, or are commercially available, such as antibodies disclosed in the following patents or patent applications: CN108473977B, CN104250302B, CN105531288B, CN105026428B, WO2008156712A1, or WO2006121168A1, or pembrolizumab, camrelizumab from Hengrui, tislelizumab from BeiGene, sintilimab from Innovent, toripalimab from Junshi Biosci, zimberelimab from Gloria Biosci, penpulimab from Chiatai tianqing, pucotenlimab from Lepu Biopharma, serplulimab from Henlius Biotech, or nivolumab from BMS.

In some embodiments, the applicable antibodies herein are the antibodies of IgG1 isotype, or the antibodies of IgG2 isotype, or the antibodies of IgG3 isotype, or the antibodies of IgG4 isotype.

In some embodiments, the applicable antibodies herein are monoclonal antibodies. In some embodiments, the applicable antibodies herein are humanized. In some embodiments, the applicable antibodies herein are human antibodies. In some embodiments, the applicable antibodies herein are chimeric antibodies.

In one embodiment, the applicable antibodies herein are full-length antibodies.

In one embodiment, the applicable antigen-binding fragment of the antibody herein is selected from the following antibody fragments: Fab, Fab′, Fab′-SH, Fv, single-chain antibody (e.g., scFv), (Fab′)2, single-domain antibodies such as VHH, dAb (domain antibodies), or linear antibodies, or half-antibodies. In some embodiments, the applicable antigen-binding fragments herein include but are not limited to, VHH, Fv molecules, scFv molecules, Fab molecules, and F(ab′)2 molecules.

In one embodiment herein, the described anti-PD-1 antibody or antigen-binding fragment thereof is an anti-PD-1 antibody or antigen-binding fragment thereof disclosed in CN108473977B, CN104250302B, CN105531288B, CN105026428B, WO2008156712A1, or WO2006121168A1. In one embodiment, the described anti-PD-1 antibody or antigen-binding fragment thereof comprises one or more CDRs (preferably 3 CDRs, i.e., HCDR1, HCDR2H, and HCDR3; or LCDR1, LCDR2, and LCDR3, more preferably 6 CDRs, i.e., HCDR1, HCDR2, HCDR3, LCDR1, LCDR2, and LCDR3) of the anti-PD-1 antibody or antigen-binding fragment thereof disclosed in CN108473977B, CN104250302B, CN105531288B, CN105026428B, WO2008156712A1, or WO2006121168A1, or comprises the VH and/or VL of the anti-PD-1 antibody or antigen-binding fragment thereof disclosed in CN108473977B, CN104250302B, CN105531288B, CN105026428B, WO2008156712A1, or WO2006121168A1, or comprises the heavy chains and/or light chains of the said antibodies.

In one embodiment herein, the described anti-PD-1 antibody or antigen-binding fragment thereof is pembrolizumab, camrelizumab, tislelizumab, toripalimab, sintilimab, zimberelimab, penpulimab, pucotenlimab, serplulimab, or nivolumab, or antigen-binding fragment thereof. In one embodiment, the described anti-PD-1 antibody or antigen-binding fragment thereof comprises one or more CDRs (preferably 3 CDRs, i.e., HCDR1, HCDR2H, and HCDR3; or LCDR1, LCDR2, and LCDR3, more preferably 6 CDRs, i.e., HCDR1, HCDR2, HCDR3, LCDR1, LCDR2, and LCDR3) of pembrolizumab, camrelizumab, tislelizumab, toripalimab, sintilimab, zimberelimab, penpulimab, pucotenlimab, serplulimab, or nivolumab, or antigen-binding fragment thereof, or includes the VH and/or VL of pembrolizumab, camrelizumab, tislelizumab, toripalimab, sintilimab, zimberelimab, penpulimab, pucotenlimab, serplulimab, or nivolumab, or antigen-binding fragment thereof, or includes the heavy chains and/or light chains of the said antibodies.

In some embodiments, the described anti-PD-1 antibody or antigen-binding fragment thereof comprises three complementary determining regions from the variable region of a heavy chain (HCDRs), namely HCDR1, HCDR2, and HCDR3. In some embodiments, the described anti-PD-1 antibody or antigen-binding fragment thereof comprises three complementary determining regions from the variable region of a light chain (LCDRs), namely LCDR1, LCDR2, and LCDR3. In some embodiments, the described anti-PD-1 antibody or antigen-binding fragment thereof comprises three complementary determining regions from the variable region of a heavy chain (HCDRs) and three complementary determining regions from the variable region of a light chain (LCDRs).

In some aspects, the described anti-PD-1 antibody or antigen-binding fragment thereof comprises the variable region of a heavy chain (VH). In some aspects, the described anti-PD-1 antibody or antigen-binding fragment thereof comprises the variable region of a light chain (VL). In some aspects, the described anti-PD-1 antibody or antigen-binding fragment thereof comprises both the variable region of a heavy chain (VH) and the variable region of a light chain (VL). In some embodiments, the described variable region of a heavy chain comprises three complementary determining regions (CDRs) from the variable region of a heavy chain, namely HCDR1, HCDR2, and HCDR3. In some embodiments, the described variable region of a light chain comprises three complementary determining regions (CDRs) from the variable region of a light chain, namely LCDR1, LCDR2, and LCDR3.

In some embodiments, the described anti-PD-1 antibody or antigen-binding fragment thereof comprises the antibody heavy chain. In some embodiments, the described anti-PD-1 antibody heavy chain comprises the variable region of a heavy chain and the constant region of a heavy chain. In some embodiments, the anti-PD-1 antibody or antigen-binding fragment thereof applicable to the present invention comprises the antibody light chain. In some embodiments, the anti-PD-1 antibody light chain herein comprises the variable region of a light chain and the constant region of a light chain. In some embodiments, the anti-PD-1 antibody or antigen-binding fragment thereof applicable to the present invention comprises both the heavy chain and the light chain.

In some embodiments, the variable region of a heavy chain is the variable region of a heavy chain of any antibody disclosed in CN108473977B, CN104250302B, CN105531288B, CN105026428B, WO2008156712A1, or WO2006121168A1, or of pembrolizumab, camrelizumab, tislelizumab, toripalimab, sintilimab, zimberelimab, penpulimab, pucotenlimab, serplulimab, or nivolumab. In some embodiments, the variable region of a light chain is the variable region of a light chain of any antibody disclosed in CN108473977B, CN104250302B, CN105531288B, CN105026428B, WO2008156712A1, or WO2006121168A1, or of pembrolizumab, camrelizumab, tislelizumab, toripalimab, sintilimab, zimberelimab, penpulimab, pucotenlimab, serplulimab, or nivolumab. In some embodiments, the CDR1, CDR2, or CDR3 from the variable region of a heavy chain is the CDR1, CDR2, or CDR3 from the variable region of a heavy chain of any antibody disclosed in CN108473977B, CN104250302B, CN105531288B, CN105026428B, WO2008156712A1, or WO2006121168A1, or of pembrolizumab, camrelizumab, tislelizumab, toripalimab, sintilimab, zimberelimab, penpulimab, pucotenlimab, serplulimab, or nivolumab. In some embodiments, the CDR1, CDR2, or CDR3 from the variable region of a light chain is the CDR1, CDR2, or CDR3 from the variable region of a light chain of any antibody disclosed in CN108473977B, CN104250302B, CN105531288B, CN105026428B, WO2008156712A1, or WO2006121168A1, or of pembrolizumab, camrelizumab, tislelizumab, toripalimab, sintilimab, zimberelimab, penpulimab, pucotenlimab, serplulimab, or nivolumab. In some embodiments, the heavy chain is the heavy chain of any antibody disclosed in CN108473977B, CN104250302B, CN105531288B, CN105026428B, WO2008156712A1, or WO2006121168A1, or of pembrolizumab, camrelizumab, tislelizumab, toripalimab, sintilimab, zimberelimab, penpulimab, pucotenlimab, serplulimab, or nivolumab. In some embodiments, the light chain is the light chain of any antibody disclosed in CN108473977B, CN104250302B, CN105531288B, CN105026428B, WO2008156712A1, or WO2006121168A1, or of pembrolizumab, camrelizumab, tislelizumab, toripalimab, sintilimab, zimberelimab, penpulimab, pucotenlimab, serplulimab, or nivolumab.

In some embodiments, the anti-PD-1 antibody or antigen-binding fragment thereof herein comprises the CDR1, CDR2, and CDR3 from the variable region of a heavy chain, as well as the CDR1, CDR2, and CDR3 from the variable region of a light chain, from any antibody disclosed in CN108473977B, CN104250302B, CN105531288B, CN105026428B, WO2008156712A1, or WO2006121168A1, or from pembrolizumab, camrelizumab, tislelizumab, toripalimab, sintilimab, zimberelimab, penpulimab, pucotenlimab, serplulimab, or nivolumab.

In some embodiments, the anti-PD-1 antibody or antigen-binding fragment thereof herein comprises the variable region of a heavy chain and the variable region of a light chain from any antibody disclosed in CN108473977B, CN104250302B, CN105531288B, CN105026428B, WO2008156712A1, or WO2006121168A1, or from pembrolizumab, camrelizumab, tislelizumab, toripalimab, sintilimab, zimberelimab, penpulimab, pucotenlimab, serplulimab, or nivolumab.

In some embodiments, the anti-PD-1 antibody or antigen-binding fragment thereof herein comprises the heavy chain and the light chain from any antibody disclosed in CN108473977B, CN104250302B, CN105531288B, CN105026428B, WO2008156712A1, or WO2006121168A1, or from pembrolizumab, camrelizumab, tislelizumab, toripalimab, sintilimab, zimberelimab, penpulimab, pucotenlimab, serplulimab, or nivolumab.

In some embodiments, the anti-PD-1 antibody or antigen-binding fragment thereof herein comprises the CDR1, CDR2, and CDR3 of the variable region of a heavy chain, as well as the CDR1, CDR2, and CDR3 of the variable region of a light chain, from pembrolizumab.

In some embodiments, the anti-PD-1 antibody or antigen-binding fragment thereof herein comprises the variable region of a heavy chain and the variable region of a light chain from pembrolizumab.

In some embodiments, the anti-PD-1 antibody or antigen-binding fragment thereof herein comprises the heavy chain and the light chain from pembrolizumab.

In some embodiments, the anti-PD-1 antibody or antigen-binding fragment thereof herein comprises the CDR1, CDR2, and CDR3 of the variable region of a heavy chain, as well as the CDR1, CDR2, and CDR3 of the variable region of a light chain, from camrelizumab.

In some embodiments, the anti-PD-1 antibody or antigen-binding fragment thereof herein comprises the variable region of a heavy chain and the variable region of a light chain from camrelizumab.

In some embodiments, the anti-PD-1 antibody or antigen-binding fragment thereof herein comprises the heavy chain and the light chain from camrelizumab.

In some embodiments, the anti-PD-1 antibody or antigen-binding fragment thereof herein comprises the CDR1, CDR2, and CDR3 of the variable region of a heavy chain, as well as the CDR1, CDR2, and CDR3 of the variable region of a light chain, from tislelizumab.

In some embodiments, the anti-PD-1 antibody or antigen-binding fragment thereof herein comprises the variable region of a heavy chain and the variable region of a light chain from tislelizumab.

In some embodiments, the anti-PD-1 antibody or antigen-binding fragment thereof herein comprises the heavy chain and the light chain from tislelizumab.

In some embodiments, the anti-PD-1 antibody or antigen-binding fragment thereof herein comprises the CDR1, CDR2, and CDR3 of the variable region of a heavy chain, as well as the CDR1, CDR2, and CDR3 of the variable region of a light chain, from sintilimab.

In some embodiments, the anti-PD-1 antibody or antigen-binding fragment thereof herein comprises the variable region of a heavy chain and the variable region of a light chain from sintilimab.

In some embodiments, the anti-PD-1 antibody or antigen-binding fragment thereof herein comprises the heavy chain and the light chain from sintilimab.

In some embodiments, the anti-PD-1 antibody or antigen-binding fragment thereof herein comprises the CDR1, CDR2, and CDR3 of the variable region of a heavy chain, as well as the CDR1, CDR2, and CDR3 of the variable region of a light chain, from toripalimab.

In some embodiments, the anti-PD-1 antibody or antigen-binding fragment thereof herein comprises the variable region of a heavy chain and the variable region of a light chain from toripalimab.

In some embodiments, the anti-PD-1 antibody or antigen-binding fragment thereof herein comprises the heavy chain and the light chain from toripalimab.

In some embodiments, the anti-PD-1 antibody or antigen-binding fragment thereof herein comprises the CDR1, CDR2, and CDR3 of the variable region of a heavy chain, as well as the CDR1, CDR2, and CDR3 of the variable region of a light chain, from zimberelimab.

In some embodiments, the anti-PD-1 antibody or antigen-binding fragment thereof herein comprises the variable region of a heavy chain and the variable region of a light chain from zimberelimab.

In some embodiments, the anti-PD-1 antibody or antigen-binding fragment thereof herein comprises the heavy chain and the light chain from zimberelimab.

In some embodiments, the anti-PD-1 antibody or antigen-binding fragment thereof herein comprises the CDR1, CDR2, and CDR3 of the variable region of a heavy chain, as well as the CDR1, CDR2, and CDR3 of the variable region of a light chain, from penpulimab.

In some embodiments, the anti-PD-1 antibody or antigen-binding fragment thereof herein comprises the variable region of a heavy chain and the variable region of a light chain from penpulimab.

In some embodiments, the anti-PD-1 antibody or antigen-binding fragment thereof herein comprises the heavy chain and the light chain from penpulimab.

In some embodiments, the anti-PD-1 antibody or antigen-binding fragment thereof herein comprises the CDR1, CDR2, and CDR3 of the variable region of a heavy chain, as well as the CDR1, CDR2, and CDR3 of the variable region of a light chain, from pucotenlimab.

In some embodiments, the anti-PD-1 antibody or antigen-binding fragment thereof herein comprises the variable region of a heavy chain and the variable region of a light chain from pucotenlimab.

In some embodiments, the anti-PD-1 antibody or antigen-binding fragment thereof herein comprises the heavy chain and the light chain from pucotenlimab.

In some embodiments, the anti-PD-1 antibody or antigen-binding fragment thereof herein comprises the CDR1, CDR2, and CDR3 of the variable region of a heavy chain, as well as the CDR1, CDR2, and CDR3 of the variable region of a light chain, from serplulimab.

In some embodiments, the anti-PD-1 antibody or antigen-binding fragment thereof herein comprises the variable region of a heavy chain and the variable region of a light chain from serplulimab.

In some embodiments, the anti-PD-1 antibody or antigen-binding fragment thereof herein comprises the heavy chain and the light chain from serplulimab.

In some embodiments, the anti-PD-1 antibody or antigen-binding fragment thereof herein comprises the CDR1, CDR2, and CDR3 of the variable region of a heavy chain, as well as the CDR1, CDR2, and CDR3 of the variable region of a light chain, from nivolumab.

In some embodiments, the anti-PD-1 antibody or antigen-binding fragment thereof herein comprises the variable region of a heavy chain and the variable region of a light chain from nivolumab.

In some embodiments, the anti-PD-1 antibody or antigen-binding fragment thereof herein comprises the heavy chain and the light chain from nivolumab.

In some embodiments, linkers can be used to attach one or more antibodies or antigen-binding fragments thereof onto red blood cells (e.g., onto membrane proteins of red blood cells), forming antibody-erythrocyte conjugates. In some embodiments, the linker is a divalent liner. In some embodiments, the antibody-erythrocyte conjugate can be prepared using the linker with reactive functional groups, which are used to covalently link to red blood cells and antibodies. In some embodiments, the linker reacts with the nucleophilic groups of the antibody or antigen-binding fragment thereof, as well as with the nucleophilic groups of the membrane proteins of the red blood cell to form covalent bonds. For example, in some embodiments, the thiol group of cysteine residues on the membrane proteins of the red blood cell can form chemical bonds with the reactive functional groups of the linker or the antibody-linker intermediate to prepare the antibody-erythrocyte conjugate.

The nucleophilic groups on antibodies or membrane proteins of red blood cells include but are not limited to: (i) N-terminal amino groups, (ii) side-chain amino groups, such as lysine, (iii) side-chain thiol groups, such as cysteine, and (iv) sugar hydroxyl or amino groups (in the case of glycosylated antibodies). Amino, thiol, and hydroxyl groups are nucleophilic and capable of reacting with electrophilic groups on the following linker moieties and linker reagents to form covalent bonds, including: (i) active esters, such as NHS esters, HOBt esters, haloformates, and acid halides; (ii) alkyl and benzyl halides, such as halogenated acetamides; and (iii) aldehydes, ketones, carboxyl groups, and maleimide groups.

In some embodiments, the nucleophilic groups on the antibodies are N-terminal amino groups or side-chain amino groups (such as the amino groups of lysine), and/or the nucleophilic groups on the membrane proteins of the red blood cell are side-chain thiol groups, such as the thiol groups resulting from the reduction of cysteine.

In one embodiment, the linker has functional groups that can react with the thiol groups on cysteine residues present on the membrane proteins of RBCs to form a covalent bond to attach to the RBCs. Non-limiting examples of such reactive functional groups include maleimide, haloacetamides, α-haloacetyl, active esters such as succinimidyl esters, 4-nitrophenyl esters, pentafluorophenyl esters, tetrafluorophenyl esters, anhydrides, acyl chlorides, sulfonyl chlorides, isothiocyanates, and isothiocyanates, preferably, maleimide.

In some embodiments, the linker has functional groups that can react with the amino groups (—NH₂) on lysine in the antibody structure to attach to the antibody. Non-limiting examples of such reactive functional groups include but are not limited to, active esters, such as NHS esters, HOBt esters, haloformates, and acid halides, preferably, N-hydroxysuccinimide ester (NHS ester).

The linker may comprise one or more linker components. Exemplary linker components include 6-maleimidocaproyl (“MC”), maleimidopropionyl (“MP”), p-aminobenzyloxycarbonyl (“PAB”), NHS esters such as N-succinimidyl 4-(2-pyridyldithio) pentanoate (“SPP”) and 4-(N-maleimidomethyl) cyclohexane-1-carboxylate (“MCC”). Various linker components are known in the field.

In some embodiments, the linker can attach to the red blood cell (RBC) by reacting with the thiol groups of free cysteine residues on the membrane proteins of the red blood cell and can attach to the antibody (Ab) by reacting with the amino groups (—NH₂) on the lysine in the antibody structure.

Exemplary linkers include but are not limited to:
bis-maleimido-trioxyethylene glycol (BMPEO), N-(β-maleimidopropyloxy)-N-hydroxysuccinimide ester (BMPS), N-(ε-maleimidocaproyloxy) succinimide ester (EMCS), N-[γ-maleimidobutyryloxy] succinimide ester (GMBS), 1,6-hexane-bis-vinylsulfone (HBVS), succinimidyl 4-(N-maleimidomethyl) cyclohexane-1-carboxy-(6-amidocaproate) (LC-SMCC), m-maleimidobenzoyl-N-hydroxysuccinimide ester (MBS), 4-(4-N-maleimidophenyl) butyryl hydrazide (MPBH), succinimidyl 3-(bromoacetamido) propionate (SBAP), succinimidyl iodoacetate (SIA), succinimidyl (4-iodoacetyl) aminobenzoate (SIAB), succinimidyl 3-(2-pyridyldithio) propionate (SPDP), N-succinimidyl-4-(2-pyridyldithio) pentanoate (SPP), succinimidyl 4-(N-maleimidomethyl) cyclohexane-1-carboxylate (SMCC), succinimidyl 4-(p-maleimidophenyl) butyrate (SMPB), succinimidyl 6-[(β-maleimidopropionamido) hexanoate] (SMPH), iminothiolane (IT), Sulfo-EMCS, Sulfo-GMBS, Sulfo-KMUS, Sulfo-MBS, Sulfo-SIAB, Sulfo-SMCC, and Sulfo-SMPB, and succinimidyl (4-vinylsulfonyl)benzoate (SVSB); also include bis-maleimide reagents: dithiobismaleimidoethane (DTME), 1,4-bismaleimidobutane (BMB), 1,4-bismaleimidyl-2,3-dihydroxybutane (BMDB), bismaleimidohexane (BMH), bismaleimidoethane (BMOE), BM (PEG) 2 (as shown below), and BM (PEG) 3 (as shown below); bis-functional derivatives of imidoesters (such as dimethyl adipimidate hydrochloride), active esters (such as disuccinimidyl suberate), aldehydes (such as glutaraldehyde), bis-azide compounds (such as bis (p-azidobenzoyl)ethylenediamine), bis-diazo derivatives (such as bis-(p-diazobenzoyl)-ethylenediamine), diisocyanates (such as tolylene-2,6-diisocyanate), and bis-active fluoride compounds (such as 1,5-difluoro-2,4-dinitrobenzene). In some embodiments, bis-maleimide reagents allow the thiol groups of cysteine in the antibody to be connected to the thiol-containing drug moiety, linker, or linker-drug intermediate. Other functional groups that react with thiol groups include but are not limited to, iodoacetamide, bromoacetamide, vinylpyridine, disulfide, pyridyl disulfide, isocyanate, and isothiocyanate.

In some embodiments, the described linker is SMCC, such as Sulfo-SMCC.

In some embodiments, the RBCs herein are natural mature red blood cells. In some embodiments, the RBCs are mature red blood cells isolated from human (for example, from adults or children) blood. In some embodiments, the RBCs are natural mature red blood cells obtained after treatment with a thiol-reducing agent, which includes cysteine residues containing thiol groups. In some embodiments, the thiol-reducing agent is TCEP.

In some embodiments, the RBCs are obtained by the following method, which includes:
(i) isolating and concentrating red blood cells from (human) whole blood, and optionally by filtering out white blood cells;
(ii) treating the red blood cells with a thiol-reducing agent (such as TCEP) to chemically modify the surface of the red blood cells; and
(iii) collecting the modified red blood cells and concentrating them.

In some embodiments, the surface chemical modification in (ii) includes the following steps:

Mixing the thiol-reducing agent with the concentrated red blood cells, wherein the concentration of the reducing agent is between 0.1 mM and 50 mM, for example, between 0.5 mM and 10.0 mM, between 0.5 mM and 5.0 mM; preferably approximately 5.0 mM.

### II. Method for Preparing Antibody-Erythrocyte Conjugates

This disclosure also provides a method for preparing antibody-erythrocyte conjugates, which includes:
(1) allowing the nucleophilic groups of the antibody or antigen-binding fragment thereof to react with a linker (such as a divalent linker) to form the antibody or antigen-binding fragment thereof connected to the linker via the covalent bond,
(2) chemically modifying the surface of natural mature red blood cells to expose nucleophilic groups of the membrane proteins on the red blood cells;
(3) mixing the antibody or antigen-binding fragment thereof connected to the linker from (1) with the red blood cells obtained from (2), allowing a covalent conjugation of the two via the linker; and
(4) collecting the antibody-erythrocyte conjugates obtained from (3).

The nucleophilic groups on antibodies or membrane proteins of red blood cells include but are not limited to: (i) N-terminal amino groups, (ii) side-chain amino groups, such as lysine, (iii) side-chain thiol groups, such as cysteine, and (iv) sugar hydroxyl or amino groups (in the case of glycosylated antibodies). Amino, thiol, and hydroxyl groups are nucleophilic and capable of reacting with electrophilic groups on the following linker moieties and linker reagents to form covalent bonds, including: (i) active esters, such as NHS esters, HOBt esters, haloformates, and acid halides; (ii) alkyl and benzyl halides, such as halogenated acetamides; and (iii) aldehydes, ketones, carboxyl groups, and maleimide groups.

In some embodiments, the nucleophilic groups on the antibodies are N-terminal amino groups or side-chain amino groups (such as the amino group of lysine), and the nucleophilic groups on the membrane proteins of the red blood cell are side-chain thiol groups, such as the thiol groups resulting from the reduction of cysteine.

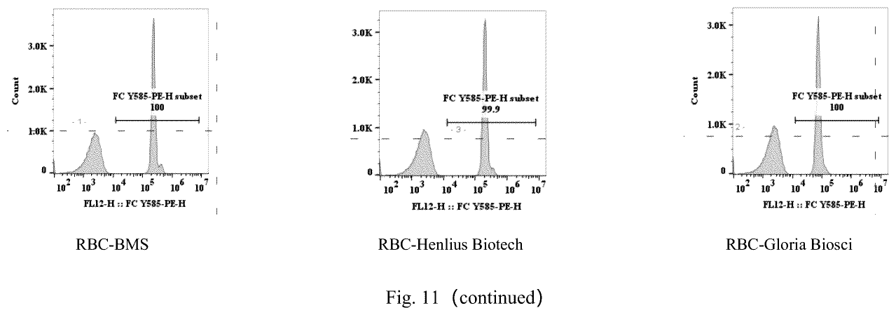

In some embodiments, the method for preparing the antibody-erythrocyte conjugates herein is shown in FIG. 10, wherein the linker represents a linker, such as SMCC (e.g., Sulfo-SMCC), and the anti-PD-1 refers to an anti-PD-1 antibody. The described method includes:
(1) connecting the amino groups on the lysine of the PD-1 antibody with the linker;
(2) chemically modifying the red blood cells to expose the thiol groups of the cysteine on the membrane proteins of the red blood cell; and
(3) conjugating the PD-1 antibody connected to the linker obtained in (1) with the cysteine on the membrane proteins of the red blood cell by the linker.

In some embodiments, the method for preparing the antibody-erythrocyte conjugates herein includes:
(1) reacting the nucleophilic groups of the antibody with a linker (e.g., a divalent linker) to form the linker attached to the antibody via the covalent bond, preferably, the described divalent linker reagent is Sulfo-SMCC, and more preferably, the described nucleophilic group is the —NH₂ of the lysine side chain,
   wherein the mentioned step (1) includes,
mixing the divalent linker with the antibody solution uniformly, wherein, for instance, the final concentration of the divalent linker in the mixture is within the range of 0.1 and 0.5 mM, for example, the final concentration is approximately 0.25 mM;
wherein, for instance, the final concentration of the antibody in the mixture is less than 0.33 mM;
preferably, mixing the mixture thoroughly, for example, at a temperature within the range of 25-35 degrees Celsius (e.g., approximately 25-approximately 30° C., e.g., approximately 30° C.), and the mixing duration is 0.5-2 hours (e.g., approximately 1 hour), for instance, placing the mixture on a rotator mixer, with a rotation speed within the range of 5-15 rpm (e.g., 10 rpm); and
subjecting the aforementioned mixture to ultrafiltration,
wherein, preferably, the buffer exchange ratio after ultrafiltration is greater than 500 times, more preferably greater than 1000 times;
wherein, the antibody concentration after ultrafiltration is approximately ≥5 mg/mL, ≥6 mg/mL, ≥7 mg/mL, ≥8 mg/mL, ≥9 mg/mL, ≥10 mg/mL, preferably ≥10 mg/ml;
preferably, this step (1) includes:
taking the 1 mg/mL of Sulfo-SMCC solution, and pipetting 21.8 μL of the Sulfo-SMCC solution into 138.2 μL of PBS buffer in an EP tube, followed by gently pipetting to mix well; pipetting 40 μL of the anti-PD-1 antibody solution into the EP tube, and mixing well to achieve the final concentration of Sulfo-SMCC of 0.25 mM. Placing the EP tube on a rotator mixer, and rotating it at 10 rpm and at a temperature of 30° C. for 1 hour to obtain the treated anti-PD-1 antibody protein solution;
adding the aforementioned treated anti-PD-1 antibody protein solution to the inner chamber of the ultrafiltration tube after the reaction is finished, then adding PBS buffer to the white line at the top of the ultrafiltration tube, following by centrifugation. After the centrifugation is finished, removing the upper part of the ultrafiltration tube, discarding the waste liquid in the lower chamber, reassembling the upper part of the ultrafiltration tube, adding PBS buffer to the white line at the top of the ultrafiltration tube, mixing well and centrifuging;
centrifuging until the volume of the upper layer is less than or equal to 100 μL (the theoretical concentration of the anti-PD-1 antibody protein solution is ≥10 mg/mL);
pipetting the upper layer of the anti-PD-1 antibody protein solution after the centrifugation is finished, and adding PBS to the volume of 100 μL (resulting in the theoretical concentration of the anti-PD-1 antibody protein solution at 10 mg/mL). The centrifugation parameters are as follows: the centrifugal force at 3800 g, the acceleration rate at 8, the deceleration rate at 8, and the temperature at 4° C.
(2) chemically modifying the surface of natural mature red blood cells to expose nucleophilic groups on the membrane proteins of the red blood cell, preferably, chemically modifying the red blood cells using the thiol-reducing agent (such as TCEP), preferably, the described nucleophilic group is the thiol group on cysteine;
wherein, the mentioned step (2) includes:
separating and concentrating red blood cells from whole blood (e.g., by leukoreduction),
mixing the thiol-reducing agent with the red blood cells uniformly, wherein the concentration of the reducing agent is between 0.1 mM and 50 mM, for example, between 0.5 mM and 10.0 mM, between 0.5 mM and 5.0 mM; preferably, approximately 5.0 mM,
preferably, mixing the mixture uniformly, for example, at a temperature within the range of 4-37 or 25-35 degrees Celsius (e.g., approximately 25° C. to approximately 30° C., e.g., approximately 30° C.), for the duration of mixture from 0.5 to 2 hours (e.g., approximately 1 hour), for instance, by placing it on a rotator mixer at the speed within the range of 5-15 rpm (e.g., 10 rpm);
centrifuging to remove the supernatant, preferably, with the centrifugal force at 500-1200 g (e.g., approximately 800 g), the centrifugation time of 2-10 minutes (e.g., approximately 3 minutes), the acceleration rate at 1-9 (e.g., approximately 9) and/or the deceleration rate at 1-7 (e.g., approximately 7);
optionally, repeating the process twice, for example, at a temperature of approximately 20° C.-30° C., for instance, at approximately 25° C.;
optionally, collecting the modified concentrated red blood cells.

Preferably, this step (2) includes:
opening the blood collection tube cap, and transferring the human blood sample from the healthy subject in the blood collection tube to the blood bag with a disposable syringe. After completion, sealing the tube with the aseptic sealing machine twice. Leukoreducing the whole blood with the leukocyte filter, and transferring the leukoreduced blood sample from the blood storage bag to the 50 mL centrifuge tube with a 20 mL disposable syringe. Centrifuging to collect red blood cells with the following centrifugation parameters set: the centrifugal force at 500 g, the centrifugation time of 5 minutes, the acceleration rate at 9, the deceleration rate at 8, and the temperature at 25° C.;
centrifuging to obtain concentrated red blood cell intermediates, with centrifugation parameters set to the centrifugal force at 500 g, the centrifugation time of 3 minutes, the acceleration rate at 9, the deceleration rate at 7, and the temperature of 25° C.;
opening the centrifuge tube, and using the disposable sterile pipette tube or the 1000 μL pipette with matching tips to discard the upper layer of liquid, then pipetting the concentrated red blood cells using the electric pipette and adding them to the reaction vessel;
pipetting 10 μL of 0.1 M TCEP and adding it to an EP tube containing 90 μL of PBS buffer solution, then mixing uniformly;
pipetting 100 μL of the aforementioned collected concentrated red blood cells and adding them to the aforementioned solution, then mixing uniformly to achieve the final concentration of TCEP at 5.0 mM;
placing the EP tube on a rotator mixer at 10 rpm and rotating at 30° C. for 1 hour;
centrifuging to remove the supernatant after the reaction is finished, then adding PBS buffer solution at a volume 5 times that of concentrated red blood cells, mixing well, and centrifuging to remove the supernatant; repeating this process twice, with centrifugation parameters to the centrifugal force at 800 g, the centrifugation time of 3 minutes, the acceleration rate at 9, the deceleration rate at 7, and the temperature at 25° C.;
collecting the modified concentrated red blood cells.
(3) mixing the antibody connected to the linker from (1) with the red blood cells obtained from (2), allowing a covalent conjugation of the two via the linker;
wherein, the mentioned step (3) includes:
mixing the antibody connected to the linker from (1) with the red blood cells obtained from (2), preferably,
mixing the mixture uniformly, for example, at a temperature within the range of 25-35 degrees Celsius (e.g., approximately 25° C. to approximately 30° C., e.g., approximately 30° C.), for the duration of mixture from 0.5 to 2 hours (e.g., approximately 1 hour), for instance, by placing it on a rotator mixer at the speed within the range of 5-15 rpm (e.g., 10 rpm);
preferably, this step (3) includes:
adding 100 μL of the modified concentrated RBCs obtained from step (2) to 100 μL of the ultrafiltrated anti-PD-1 antibody protein solution obtained from step (1), followed by gently pipetting to mix well;
placing the EP tube on a rotator mixer and agitating at 10 rpm and 30° C. for 1 hour;
(4) collecting the antibody-erythrocyte conjugates obtained from (3),
centrifuging the mixture obtained from step (3) to remove the supernatant, optionally, repeating the process twice, for example, at a temperature of 20-30 degrees Celsius, for example, at approximately 25° C.
preferably, this step (4) includes:
centrifuging the mixture obtained from step (3) to remove the supernatant, then adding physiological saline at a volume 9 times that of the concentrated RBCs, mixing well and centrifuging to remove the supernatant; repeating this process twice with the following centrifugation parameters: the centrifugal force at 800 g, the centrifugation time of 3 minutes, the acceleration rate at 9, the deceleration rate at 7, and the temperature at 25° C. collecting the engineered red blood cells.
optionally, the described method also includes step (5): preserving the engineered red blood cells obtained from step (4),
preferably, the mentioned step (5) includes:
pipetting 100 μL of physiological saline and 50 μL of erythrocyte preservation solution (physiological saline equivalent to the volume of concentrated red blood cells and 0.5 times the volume of erythrocyte preservative solution (such as CPDA-1 erythrocyte preservation solution)), adding to the engineered red blood cells, followed by gently pipetting to mix well, resulting in the engineered red blood cell preparation solution, which is then added to a packaging container, and storing the packaged preparation at 4° C. for future use.

In some embodiments, during the centrifugation in the mentioned steps, the centrifugal force is 500˜1200 g, the centrifugation time is 2˜10 minutes, the acceleration rate is 1˜9 and/or the deceleration rate is 1˜7.

Therefore, in one embodiment, the present invention also relates to the antibody-erythrocyte conjugate prepared by the aforementioned method.

### IV. Therapy

In one aspect, the present invention provides a pharmaceutical composition or preparation comprising the antibody-erythrocyte conjugates. These compositions or preparations may also optionally comprise suitable pharmaceutical adjuvants, such as pharmaceutical carriers and pharmaceutical excipients known in the art, including buffers.

In one aspect, the present invention provides a blood preparation comprising the antibody-erythrocyte conjugates herein, such as a human blood preparation. In some embodiments, the human blood preparation herein contains 10-1000 μg/mL of the antibody-erythrocyte conjugates, for example, 10-500 μg/mL of the antibody-erythrocyte conjugates, for example, at 50 μg/mL, 60 μg/mL, 70 μg/mL, 80 μg/mL, 90 μg/mL, 100 μg/mL, 150 μg/mL, 200 μg/mL, 250 μg/mL, 300 μg/mL, 350 μg/mL, 400 μg/mL, 450 μg/mL, or 500 μg/mL or more, or within any range of the said values. In some embodiments, the blood preparation herein is a leukoreduced blood preparation, i.e., a blood preparation from which leukocytes have been removed. In some embodiments, the blood preparation herein is a leukoreduced human blood preparation.

In some embodiments, the blood preparation herein can be an allogeneic blood preparation, such as an allogeneic human blood preparation. In some embodiments, the red blood cells in the blood preparation are derived from healthy donors.

In some embodiments, the blood preparation herein can be an autologous blood preparation, such as an autologous human blood preparation. In some embodiments, the red blood cells in the blood preparation are derived from the subject to be treated.

In one aspect, the present invention also provides a combination product (e.g., a pharmaceutical combination product) comprising the antibody-erythrocyte conjugates herein, as well as one or more other therapeutic agents. The combination product herein can be used in the therapeutic methods herein.

The present invention also provides a kit comprising the mentioned combination product, for example, the described kit in the same packaging includes:
a first container containing the antibody-erythrocyte conjugates herein or a pharmaceutical composition or a preparation comprising the same;
a second container containing one or more other therapeutic agents or a pharmaceutical composition or a preparation comprising the same.

In some embodiments, the mentioned therapeutic agents are selected from any substances effective in cancer treatment, including chemotherapeutic agents, other antibodies, cytotoxic agents, vaccines, small molecule drugs, or immunomodulators (such as immunosuppressants or immune agonists). Preferably, the therapeutic agents are selected from tumor vaccines, immune checkpoint inhibitor antibodies, or immune agonist antibodies.

In one aspect, the present invention relates to a method for preventing or treating a disease in a subject, such as cancer, the method comprising administering to the subject an effective amount of the antibody-erythrocyte conjugates described herein.

The cancer can be at an early, intermediate, or advanced stage, or it can be metastatic. In some embodiments, the cancer can be a solid tumor or a hematological tumor. In some embodiments, the described cancer is a gastrointestinal tumor, such as colon cancer or colorectal cancer; or it may be lung cancer, pancreatic cancer, or breast cancer. In some embodiments, the described tumor is a tumor or cancer that is resistant or insensitive to known drugs, such as known PD-1 inhibitors, such as anti-PD-1 antibodies, for example, refractory tumors or cancers.

In some embodiments, the described cancer is the cancer characterized by having elevated protein levels and/or nucleic acid levels (e.g., increased expression) of PD-1, PD-L1, and/or PD-L2, for example, the tumor cells of the described cancer have elevated protein levels and/or nucleic acid levels (e.g., increased expression) of PD-1, PD-L1, and/or PD-L2, such as compared to the protein levels and/or nucleic acid levels of PD-1, PD-L1, and/or PD-L2 in normal cells of corresponding tissues in healthy individuals, or compared to the protein levels and/or nucleic acid levels of PD-1, PD-L1, and/or PD-L2 in normal cells of adjacent healthy tissue in the same individual.

In some embodiments, the antibody-erythrocyte conjugates herein can be used to stimulate the immune system of the host, for example, to enhance the immune response of cells. “Stimulating the immune system” may include one or more of the following: an overall increase in immune function, an increase in T cell function, an increase in B cell function, restoration of lymphocyte function, an increase in IL-2 receptor expression, an increase in T cell responsiveness, an increase in T cell activity or natural killer cell activity or lymphokine-activated killer (LAK) cell activity, an increase in T cell or natural killer cell survival, and an increase in the expression of cytotoxic effector proteins, etc.

The antibody-erythrocyte conjugates (as well as compositions, pharmaceutical compositions, preparations, and combination products, etc., comprising the same, such as blood preparations) herein can be administered by any suitable method, preferably by infusion, for example, parenteral infusion. Parenteral infusion includes intravenous or intra-arterial administration. Preferably, the antibody-erythrocyte conjugates herein are administered by intravenous or arterial infusion.

To prevent or treat a disease, the appropriate dosage of the antibody-erythrocyte conjugates herein (when used alone or in combination with one or more other therapeutic agents) will depend on the type of disease to be treated, the type of antibody, the severity and progression of the disease, whether it is administered for preventive purposes or for therapeutic purposes, previous treatments, the patient's clinical history and response to the said antibody, and the judgment of the attending physician. The described antibody is appropriately administered to the patient in a single treatment or over a series of treatments.

In some embodiments, the antibody-erythrocyte conjugates herein, or pharmaceuticals or preparations comprising the same, are administered in combination with one or more therapeutic modalities or other therapeutic agents. In some embodiments, the described therapeutic modality is radiotherapy or surgery. In some embodiments, the described therapeutic agents are selected from any substances effective in cancer treatment, including chemotherapeutic agents, other antibodies, cytotoxic agents, vaccines, small molecule drugs, or immunomodulators (such as immunosuppressants or immune agonists). Preferably, the therapeutic agents are selected from tumor vaccines, immune checkpoint inhibitor antibodies, or immune agonist antibodies.

In another aspect, the present invention also provides the use of the antibody-erythrocyte conjugates herein in the preparation of pharmaceuticals or preparations for the aforementioned methods (e.g., for treatment).

In some embodiments, the red blood cells in the antibody-erythrocyte conjugates herein are derived from the subject to be treated. In some embodiments, the method of treating a subject's disease herein includes:
(1) collecting blood from the subject to be treated;
(2) preparing the antibody-erythrocyte conjugates, for example, by the methods described herein; and
(3) administering to the subject a therapeutically effective amount of the antibody-erythrocyte conjugates.

In some embodiments, the red blood cells in the antibody-erythrocyte conjugates herein can be derived from other subjects, such as healthy subjects. In some embodiments, the antibody-erythrocyte conjugates herein can be administered to the subject to be treated as pharmaceuticals or preparations.

Any references cited herein, including patents, patent applications, and literature, are incorporated herein in their entirety.

Any and all features discussed in the preceding text herein, as well as throughout this entire application, can be combined in various embodiments herein. The following examples further illustrate the present invention, however, it should be understood that the examples are described in an illustrative rather than a limiting manner, and are not intended to, and should not, in any way limit the scope of protection herein, and those skilled in the art can make various modifications.

### EXAMPLES

### Example 1 Preparation of Red Blood Cells Targeting PD-1

### 1. Preparation of Engineered Red Blood Cells (Taking the Reaction of 100 μL of Concentrated RBCs as an Example)

### 1.1 Obtaining the Intermediate Product of Red Blood Cells

the blood collection tube cap was opened, and the human blood sample from the healthy subject in the blood collection tube was transferred to the blood bag with a disposable syringe. After completion, the tube was sealed with an aseptic sealing machine twice. The whole blood was leukoreduced with the leukocyte filter, and the leukoreduced blood sample from the blood storage bag was transferred to the 50 mL centrifuge tube with a 20 mL disposable syringe. The tube was centrifuged to collect red blood cells with the following centrifugation parameters set: the centrifugal force at 500 g, the centrifugation time of 5 minutes, the acceleration rate at 9, the deceleration rate at 8, and the temperature at 25° C.

The tube was centrifuged to obtain the concentrated red blood cell intermediates with centrifugation parameters set to the centrifugal force at 500 g, the centrifugation time of 3 minutes, the acceleration rate at 9, the deceleration rate at 7, and the temperature at 25° C. The centrifuge tube was opened, and the supernatant liquid was discarded with a disposable sterile pipette tube or a 1000 μL pipette with matching tips, then the concentrated red blood cells were pipetted using the electric pipette and added to the reaction vessel.

### 1.2 Processing of Antibody Protein

### 1.2.1 Preparation of Sulfo-SMCC

Sulfo-SMCC powder (purchased from Thermo Fisher, Cat. No. 22322) was taken out from the −20° C. refrigerator, accurately weighed to 2 mg, and placed into an EP tube, 2 mL of PBS was added, and the tube was vibrated to ensure complete dissolving of the powder, with the resulting concentration of 1 mg/mL.

### 1.2.2 Processing of Antibody Protein

1.8 μL of 1 mg/mL Sulfo-SMCC solution was pipetted into 138.2 μL of PBS buffer, followed by gently pipetting to mix well. 40 μL of the anti-PD-1 antibody solution (Keytruda®, MSD) was pipetted into an EP tube, followed by mixing well to achieve a final concentration of Sulfo-SMCC at 0.25 mM. The EP tube was placed on a rotator mixer, and rotated at 10 rpm and at a temperature of 30° C. for 1 hour to obtain the treated anti-PD-1 antibody protein solution.

The aforementioned treated PD-1 antibody protein solution was added to the inner chamber of the ultrafiltration tube after the reaction is finished, then PBS buffer was added to the white line at the top of the ultrafiltration tube, following by centrifugation. After the centrifugation was finished, the upper part of the ultrafiltration tube was removed, the waste liquid in the lower chamber was discarded, the upper part of the ultrafiltration tube was reassembled, PBS buffer was added up to the white line at the top of the ultrafiltration tube, followed by mixing well and centrifugation until the volume of the upper layer is less than or equal to 100 μL (the theoretical concentration of the anti-PD-1 antibody protein solution is ≥10 mg/mL). The upper layer of the anti-PD-1 antibody protein solution was pipetted out after the centrifugation is finished, and PBS was added to the volume of 100 μL (resulting in the theoretical concentration of the anti-PD-1 antibody protein solution at 10 mg/mL). The centrifugation parameters were as follows: the centrifugal force at 3800 g, the acceleration rate at 8, the deceleration rate at 8, and the temperature at 4° C.

### 1.3 Surface Modification of Red Blood Cells

### 1.3.1 Preparation of 0.1M TCEP

An ampoule of TCEP stock solution (purchased from Sigma-Aldrich, Cat. No. 646547) with the labeled concentration of 0.5 M was taken from the −20° C. refrigerator, and stood on ice until the solution in the vial was completely melted. After surface disinfection, the ampoule was placed in a biosafety cabinet and 1 stood for 1 minute. The ampoule was carefully open, 100 μL of TCEP stock solution was taken and added to a 2 mL EP tube, then 400 μL of PBS was added, followed by mixing well by pipetting to prepare the diluted TCEP stock solution. The diluted TCEP stock solution was pipetted with a 1 ml syringe and filtered through a 0.22 μm filter membrane to sterilize, obtaining an aseptic solution of TCEP with a concentration of 0.1 M. The aseptic TCEP solution was dispensed into sterile 1.5 mL EP tubes by 20 μL per tube. The tubes were sealed, with sample labels attached, and stored in the −20° C. refrigerator for future use.

### 1.3.2 Surface Modification of Red Blood Cells

10 μL of 0.1M TCEP was pipetted and added to an EP tube containing 90 μL of PBS buffer, followed by mixing uniformly. 100 μL of the concentrated red blood cells obtained from the aforementioned step 1 were pipetted and added to the aforementioned solution, followed by mixing uniformly to achieve the final concentration of TCEP at 5.0 mM. The EP tube was on placed on a rotator mixer and rotated at 10 rpm, 30° C. for 1 hour.

The supernatant was removed by centrifugation after the reaction was finished, then PBS buffer solution was added at a volume 5 times that of concentrated red blood cells, followed by mixing well and centrifugation to remove the supernatant. This process was repeated twice, with centrifugation parameters set to the centrifugal force at 800 g, the centrifugation time of 3 minutes, the acceleration rate at 9, the deceleration rate at 7, and the temperature at 25° C. The modified concentrated red blood cells were collected.

### 1.4 Engineering of Red Blood Cells

100 μL of the modified concentrated RBCs were added to 100 μL of the ultrafiltrated anti-PD-1 antibody protein solution, followed by pipetting gently to mix well. The EP tube was placed on a rotator mixer and agitated at 10 rpm, 30° C. for 1 hour.

After the reaction was finished, the supernatant was removed by centrifugation, then physiological saline was added at a volume 9 times that of the concentrated RBCs, followed by mixing well and centrifugation to remove the supernatant. This process was repeated twice with the following centrifugation parameters set: the centrifugal force at 800 g, the centrifugation time of 3 minutes, the acceleration rate at 9, the deceleration rate at 7, and the temperature at 25° C. The engineered red blood cells were collected.

### 2. Preservation of Engineered Red Blood Cells

100 μL of physiological saline and 50 μL of erythrocyte preservative solution (physiological saline equivalent to the volume of concentrated red blood cells and 0.5 times the volume of CPDA-1 erythrocyte preservative solution (Shandong Weigao Group)) were pipetted to add to the engineered red blood cells, followed by gently pipetting to mix well, resulting in the engineered red blood cell preparation solution, which was then added to a packaging container, and the packaged preparation was stored at 4° C. for future use.

In the following text, the Keytruda-conjugated engineered red blood cells prepared in this example are referred to as RBCs-Keytruda or RBC-Keytruda for short.

### Example 2 In Vitro Activity of Engineered Red Blood Cells

### PD-1/PD-L1 Blockade Bioassay

### 1. Experimental Principle:

When co-culturing the two types of cells, the interaction of PD-1/PD-L1 inhibits TCR signal transduction and NFAT-mediated luciferase activity. The addition of any antibody that can block either PD-1 or PD-L1 will negate the inhibitory signal, thereby activating the TCR signaling pathway and enhancing NFAT-mediated luciferase activity.

### 2. Experimental Materials

PD-1 effector cells: Jurkat T cells (Promega, J115A), stably expressing human PD-1 and NFAT-inducible luciferase.

PD-L1 aAPC/CHO-K1 cells (Promega, J109A): CHO-K1 cells, stably expressing human PD-L1 and a cell surface protein capable of activating the cognate TCR in an antigen-independent manner.

### 3. Experimental Equipments

| Equipment Name | Manufacturer | Model |
|---|---|---|
| Multimode plate reader | Molecular Devices | SpectraMax iD5 |
| 2-8° C. refrigerator | Midea | BCD207MW |
| Pipette | Germany Brand | 0.5~10 μL |
| | | 20~200 μL, |
| | | 100~1000 μL |
| Multi-channel pipette | RAININ | XLS-200 UL |
| CO₂ incubator | ESCO | CLM-170B-8-CN |
| Biosafety cabinet | ESCO | AC2-6S1 |
| Desktop high-speed | Thermo | SorvallST16R |
| refrigerated centrifuge | | |

### 4. Experimental Consumables

| Name | Manufacturer | Model |
|---|---|---|
| Test plate | CORNING | 3610 |
| Reagent reservoirs | BIOLOGIX | 25-0051 |
| Dilution plate | Greiner | 655185 |
| Pipette | BIOLOGIX | 5 mL, 10 mL, 25 mL, 50 mL |

### 5. Experimental Reagents

| Name | Manufacturer | Product Number |
|---|---|---|
| DMEM/F12 medium | Cytiva | SH30023.01 |
| Fetal Bovine Serum | GIBCO | 10270-106 |
| Hygromycin B | Invitrogen | 10687010 |
| GENETICIN (G418) | GIBCO | 10131-035 |
| 0.25% Trypsin-EDTA | GIBCO | 25200-056 |
| DPBS | SIGMA | D8537-500 ML |
| RPMI-1640 Medium | SIGMA | R8758-500 ML |
| Puromycin | MCE | HY-B1743A/CS-6857 |

### III Experimental Procedures

### 1. Preparation of Cell Lines

Cell digestion: To a T75 culture flask, which was taken as an example, was added 1 mL of trypsin, and the cell culture flask was placed in a CO₂ incubator for about 2-3 minutes to allow the cells to detach from the flask surface. Growth medium was added to the culture flask to terminate the digestion, and the cells were rinsed off with a pipette. The cell suspension from the culture flask was transferred into one disposable sterile centrifuge tube, followed by centrifugation at 1000 rpm for 5 minutes. The supernatant was discarded, and 10 mL of growth medium was added to resuspend the cells. The cell density and cell viability were determined with a cell counter. the cell density was adjusted to 4×10⁵ cells per milliliter. 100 μL of the cell suspension was added to the wells of a white flat-bottom 96-well assay plate, with a final count of 40,000 cells per well.

The assay plate was placed in the CO₂ incubator to culture overnight (16˜20 hours).

### 2. Detection Methods

Serial dilution of PD-1 antibody and red blood cell preparation samples to be tested: the fresh assay buffer (98% RPMI 1640+2% FBS) was prepared on the day of the assay. The required volume of assay buffer was determined based on the number of samples. The PD-1 antibody (Keytruda®, MSD) was diluted with the assay buffer (98% RPMI 1640+2% FBS) for a 3-fold serial dilution across 10 points, with the highest concentration dilution point at 5 μg/mL, wherein the sample was taken for each step at a volume not less than 10 μL, and mixed well by a vortex mixter after each dilution step. When the sample concentration was above 1 mg/mL, each dilution step should not exceed a 100-fold dilution.

Preparation of Jurkat-PD-1 Effector cells: the Jurkat T cells were passaged before the assay, and cultured to a density of 1×10⁶ cells/mL before the assay, ensuring that the cell viability was greater than 80%. Cell collection: the medium supernatant from the cell culture flask was transferred to a 50 mL disposable sterile centrifuge tube, and the cells were resuspended with the assay buffer at a density of 1.25×10⁶ cells/mL.

Addition of antibodies or red blood cell preparations and Jurkat-PD-1 Effector cells to the assay plate: 40 μL of the serially diluted to be tested red blood cell preparation or PD-1 antibody was added to the corresponding wells, and assay buffer was added to the blank control wells. 40 μL of Jurkat-PD-1 Effector cells were added to the wells containing PD-L1 aAPC/CHO-K1 cells, and antibodies or red blood cell preparations, as well as blank controls. the assay plate was incubated in a 37° C./5% CO₂ incubator for 4˜6 hours. Bright-Glo® luciferase assay: 40 μL of Bright-Glo® reagent was added to the wells containing cells, followed by incubation at room temperature for 8-15 minutes. The luminescent signal was detected using a luminometer or a microplate reader equipped with luminescence detection functions.

### IV. Data Analysis

Sample concentration calculation methods: data calculation and analysis were performed using data processing software. The values of % CV for the samples to be tested and the reference standard were calculated in duplicate wells. With the concentration of the standard as the X-axis and the induction fold as the Y-axis, a four-parameter logistic regression model was used to plot the standard curve and calculate the R2 value of the fitted curve. y=D+(A−D)/(1+(X/C)\hat{ }B)

The formula for calculating the relative activity of the sample is as follows: Relative binding activity=EC50 value of the reference standard/EC50 value of the test sample*100%

The formula for calculating the relative standard deviation is as follows: RSD=(SD/AVERAGE)×100%

The parallelization slope ratio=Test sample B value/Reference standard B value

The upper asymptote ratio (D value ratio)=Test sample D value/Reference standard D value

### V. Experimental Results

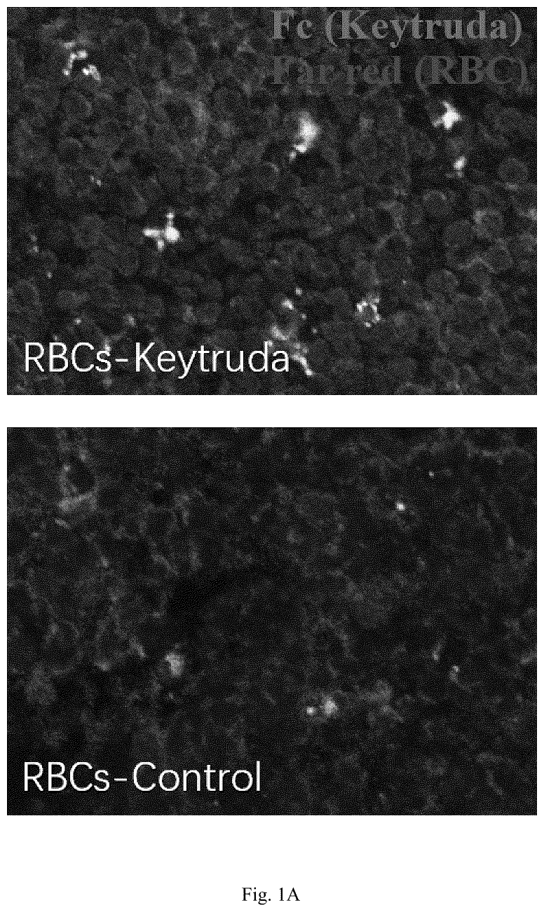

The experimental results are shown in FIG. 1B and FIG. 1C. As indicated by the PD-1/PD-L1 blockade bioassay results, RBC-Keytruda can produce an in vitro agonistic effect more rapidly at low doses of Keytruda (FIG. 1B). Under the same dosage conditions, the RBC-Keytruda preparation can significantly enhance the efficacy of the PD-1 antibody (FIG. 1C).

### Example 3 Treatment of the MC38 Model with SK-RBC

### 1. Research Objective:

The objective of this Research was to evaluate the tumoricidal efficacy of Keytruda-conjugated mouse engineered red blood cells in PD-1 humanized mice model harboring the MC38 cell tumor.

### 2. Research Methods

Murine colon cancer MC38 cells (GemPharmatech) were subcutaneously injected into PD-1 humanized mice at a dosage of 5×10⁵ cells.

In this mouse model, all experimental animals were divided into four groups:
(1) a control group, blank Control PBS;
(2) a low-dose Keytruda treatment group;
(3) a high-dose Keytruda treatment group; and
(4) a RBCs-Keytruda treatment group.

When the tumor size in the mice reached approximately 50 mm³, the animals were randomly divided into groups with initiation of medication. The tumor volume was measured every 3-4 days, and the mice were euthanized when the tumors in the control group reached 2000 mm³. The tumor volumes among the different groups of mice were compared.

### Experimental Materials:

| | Lot No. | Specifications | | Cat. No. | Manufacturer |
|---|---|---|---|---|---|
| Keytruda | S007467 | 25 mg/mL * 4 mL | | / | MSD Ireland |
| PBS | L210807 | 500 | mL | B320KJ | Shanghai |
| | | | | | BasalMedia |
| TCEP | MKCM6848 | 100 mM * 1 mL | | 646547 | Sigma |
| | | (After | | | |
| | | subpackaging) | | | |
| c57 mRBC | / | 3 | mL | / | |
| PE anti-Human IgG | 409304 | 500 | μL | B318395 | Biolegend |
| Fc Secondary Antibody | | | | | |

| | | | | |
|---|---|---|---|---|
| Sulfo-SMCC (sulfosuccinimidyl | / | 2 mg * 10 | A39268 | Thermo |
| 4-(N-maleimidomethyl)cyclohexane- | | | | Scientific |
| 1-carboxylate), No-Weigh ™ Format | | | | |

### 2.1 Preparation of Keytruda-Conjugated Mouse Engineered Red Blood Cells

Keytruda-conjugated mouse engineered red blood cells were prepared similar to Example 1, wherein

Treatment of 5 mg of Keytruda with Sulfo-SMCC (Molar Ratio of 9.3:1)

A vial of Sulfo-SMCC was taken from a −20° C. refrigerator, and 2 mL of PBS was added, followed by vibration to ensure the complete dissolving of Sulfo-SMCC, resulting in a final concentration of 1 mg/mL.

To a 2.0 mL EP tube were added samples according to the following table:

| | | | Sulfo-SMCC | Keytruda |
|---|---|---|---|---|
| Sulfo-SMCC | Keytruda | PBS | concentration | concentration |
| 250 μL | 200 μL | 1540 μL | 156.2 μM | 16.7 μM |

After incubation at 10 rpm for 1 hour, the ultrafiltration was performed twice using a 50 kDa ultrafiltration tube at a speed of 3800 g for 15 minutes, then the volume was adjusted to 250 μL.

Treatment of 1.5 mL of Concentrated mRBC with TCEP

3 mL of whole blood collected from adult PD-1 humanized mice, i.e. C57BL/6 mice, was transferred to a centrifuge, followed by centrifugation at 1000 g for 5 minutes, then the supernatant was removed. The concentrated RBC was transferred to a 15 mL centrifuge tube filled with PBS, followed by centrifugation again at the same speed and duration to wash the cells twice, and the supernatant was removed. Samples were added according to the following table.

| TCEP | mRBC | PBS | TCEP concentration |
|---|---|---|---|
| 40 μL | 1500 μL | 6460 μL | 0.5 μM |

After a reaction at 10 rpm for 1 hour, the mRBC was washed twice with PBS using the aforementioned method, and then the supernatant was removed.

Mixing the Ultrafiltrated Keytruda with 500 μL of RBC (without Adding PBS), Followed by Mixing Well at 10 Rpm for 1 Hour, and Washing Twice with PBS.

The samples were labeled with the FC-conjugated antibody at a ratio of 1:200 for 2 minutes, then flow cytometry testing was performed. Subpackaging was performed after achieving a coupling efficiency of 100%.

Subpackaging the Prepared Blood Samples into Aliquots According to a Concentration Gradient of 1:5

416 μL of RBCs-Keytruda was taken, supplemented with PBS to a final volume of 1.25 mL, and labeled as “RBCs-Keytruda”.

A solution of Keytruda with a concentration of 0.005 mg/mL was prepared using PBS to a final volume of 1.25 mL, and labeled as “Keytruda-low”.

A solution of Keytruda with a concentration of 0.05 mg/mL was prepared using PBS to a final volume of 1.25 mL, and labeled as “Keytruda-high”. 1.25 ml of PBS was taken and labeled as “Control PBS”.

### 2.2 Experimental Methods

### Experimental Animals:

The animals used in this experiment were PD-1 humanized C57BL/6 female mice, purchased from GemPharmatech. The initial experimental age for all mice was 6 to 8 weeks old.

### Drug Administration and Grouping Design:

Administration route and Administration by intravenous injections via the tail vein method:
Rationale for the The transfusion method for the engineered red blood cell selection of products was based on the transfusion method for concentrated administration route: red blood cell products as stipulated in the “Guidelines on the Quality Monitoring of Whole Blood and Blood Components”.
Administration frequency Once every three days for a total of six injections (q3d×6) and duration:
Administration time: 16:00˜17:00
Administration volume: 200 μL
Administration rate: 25 μL/s, using a syringe to control the rate of administration.

| | Number of | Mode of | Administration |
|---|---|---|---|
| Groups | Animals | Administration | Frequency |
| G1: Control | 6 | Intravenous | q3 d |
| group, PBS | | injections via | |
| | | the tail vein | |
| G2: Keytruda | 6 | Intravenous | q3 d |
| low-dose | | injections via | |
| treatment group | | the tail vein | |
| G3: Keytruda | 6 | Intravenous | q3 d |
| high-dose | | injections via | |
| treatment group | | the tail vein | |
| G4: RBCs- | 6 | Intravenous | q3 d |
| Keytruda group | | injections via | |
| | | the tail vein | |

Wherein the MC38 cell was selected as the tumor cell line, which facilitated subcutaneous tumors formation in C57 BL/6 mice.

### 3. Experimental Results

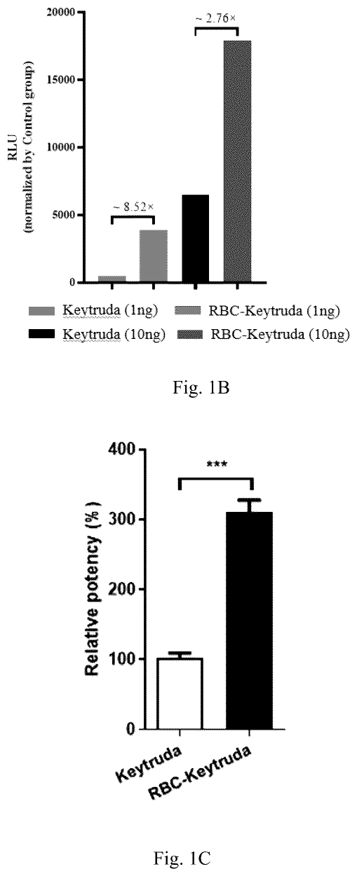

The results were shown in FIG. 2. Based on the statistical analysis of the tumor volume data during the drug efficacy process, compared to the G1 (PBS) control group: the Keytruda group (G2, 0.05 mg/kg) did not exhibit significant tumor growth inhibition; the Keytruda group (G3, 0.5 mg/kg) began to show significant tumor growth inhibition from D7 (the seventh day) after administration, reaching the peak value of tumor inhibition rate (TGItv=78.02%) on the D24 (the 24^{th} day) of observation, and showed a significant statistical difference (**P<0.01). Subsequently, the TGI gradually declined, with the tumor inhibition rate on D30 (the 30^{th} day) being TGItv=75.53%.

The RBCs-Keytruda group (G4, 0.5 mg/kg) began to show significant tumor growth inhibition starting from D7 post-administration, reaching the peak value of the tumor growth inhibition rate on D27 (the 27^{th} day) of observation (TGItv=91.09%), and showed a significant statistical difference (***P<0.001). Subsequently, the TGI gradually declined, with the tumor inhibition rate on D30 (the 30^{th} day) being TGItv=90.44%. Mice treated with Keytruda-conjugated mouse engineered red blood cells did not show significant statistical differences in body weight changes across groups. In summary, the high-dose administration group of Keytruda-conjugated mouse engineered red blood cells had a significant anti-tumor effect on tumor-harboring mice with the MC38 mouse colon cancer cell line.

TGITV (relative tumor growth inhibition rate) calculation formula:

$$R T {V}_{n} = \frac{- {V}_{n t}}{{V}_{n 0}}$$

$$TGI = ( 1 - \frac{mean {RTV}_{t r e a t}}{mean {RTV}_{v e h i c l e}} ) \times 100 \%$$

Vₙₜ: the tumor volume of mouse numbered n on day t;
Vₙ₀: the tumor volume of mouse numbered n on day 0;
RTVₙ: the relative tumor volume of mouse numbered n on day t;
mean RTVₜᵣₑₐₜ: average value of RTV of the administration group;
mean RTV_{vehicle}: average value of RTV of the control group (group 1);
wherein the tumor volume was measured using a caliper.

### Example 4 Treatment of the KP Model with SK-RBC

### 1. Research Objective

The objective of this research was to evaluate the tumoricidal efficacy of Keytruda-conjugated mouse engineered red blood cells (RBC-Keytruda, prepared as described in Example 3) in PD-1 humanized mice model harboring the KP cell tumor.

### 2. Research Methods

The mouse lung cancer KP (KRAS G12D, P53−/−) cells (Shanghai Cell Bank) were subcutaneously injected into a mouse with the cell number of 1×10⁶. In this mouse model, all experimental animals were divided into three groups: (1) a control group, with blank Control RBCs; (2) a RBC-Keytruda high-dose treatment group; (3) a Keytruda monotherapy group. When the tumor size in the mice reached approximately 50 mm³, the animals were randomly divided into groups with initiation of medication. The tumor volume was measured every 3-4 days, and the mice were euthanized when the tumors in the control group reached 2000 mm³. The tumor volumes were compared between the two groups of mice.

### 3. Experimental Animals

The animals used in this experiment were PD-1 humanized female C57BL/6 mice, purchased from GemPharmatech. The initial experimental age for all mice was 6 to 8 weeks old.

### 4. Animal Administration Methods

Administration route and Administration by intravenous injections via the tail vein method:
Rationale for the selection of The transfusion method for the engineered red blood cell administration route: products was based on the transfusion method for concentrated red blood cell products as stipulated in the “Guidelines on the Quality Monitoring of Whole Blood and Blood Components”.
Administration frequency and Once every three days for a total of six injections (q3d×6) duration:
Administration time: 16:00˜17:00
Administration volume: 200 μL
Administration rate: 25 μL/s, using a syringe to control the rate of administration.

### 5. Experimental Animal Grouping

| | Number of | Mode of | Administration |
|---|---|---|---|
| Groups | Animals | Administration | Frequency |
| (1) Control group, | 5 | Intravenous injections | q3 d |
| Control RBC | | via the tail vein | |
| (2) RBC-Keytruda | 5 | Intravenous injections | q3 d |
| treatment group | | via the tail vein | |
| (3) Keytruda | 5 | Intravenous injections | q3 d |
| monotherapy group | | via the tail vein | |

Wherein the KP cell was selected as the tumor cell line, which facilitated subcutaneous tumors formation in C57/BL6 mice.

### 6. Experimental Results

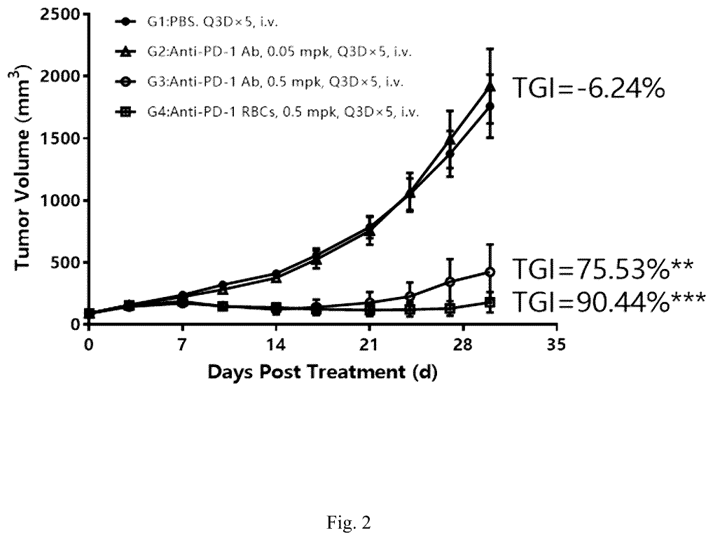

As shown in FIG. 3, the survival status of the mice was monitored at different time points. The Keytruda monotherapy group, due to poor health conditions of the mice, resulted in the death of all mice by the 31^{st} day of the experiment. In contrast, all mice of the control group and the RBC-Keytruda treatment group were still alive after 39 days of the experiment. The pharmacokinetics in vivo indicated that after Keytruda administration, there was a high concentration in the peripheral blood, leading to certain tissue toxicities, such as intestinal toxicity. However, with the administration of the RBC-Keytruda herein, its concentration in the peripheral blood was low and primarily distributed in the vascular and the liver and spleen system, therefore, resulting in less tissue toxicity.

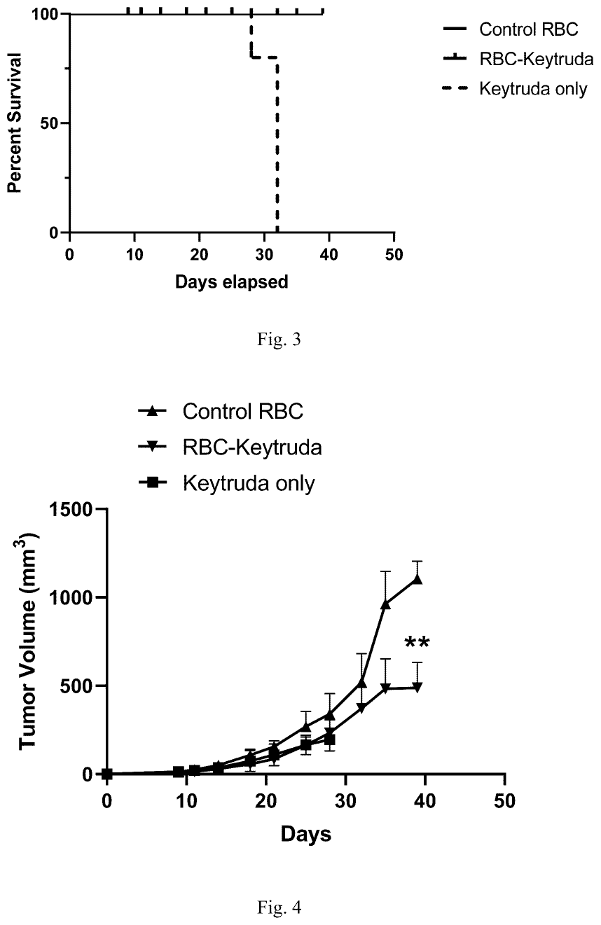

As shown in FIG. 4, after treatment with Keytruda-conjugated mouse engineered red blood cells (RBC-Keytruda), on the 39^{th} day following the injection of KP tumor cells, the mice tumor volume was measured using a caliper. The average tumor size in the control group mice was 1103.95 mm³, while the average tumor size in the RBC-Keytruda treatment group was 488.32 mm³. The tumor size in the treatment group was significantly smaller than that in the control group (**p<0.01), with a tumor growth inhibition rate (TGItw) of 55.76%.

Tumor growth inhibition rate (TGItw) calculation formula:

$$TGItw = ( 1 - meanTW treat / meanTW vehicle ) \times 100 \%$$

meanTWtreat: the average tumor weight of the mice in the treatment group at the endpoint of treatment
meanTW vehicle: the average tumor weight of the mice in the control group at the endpoint of treatment

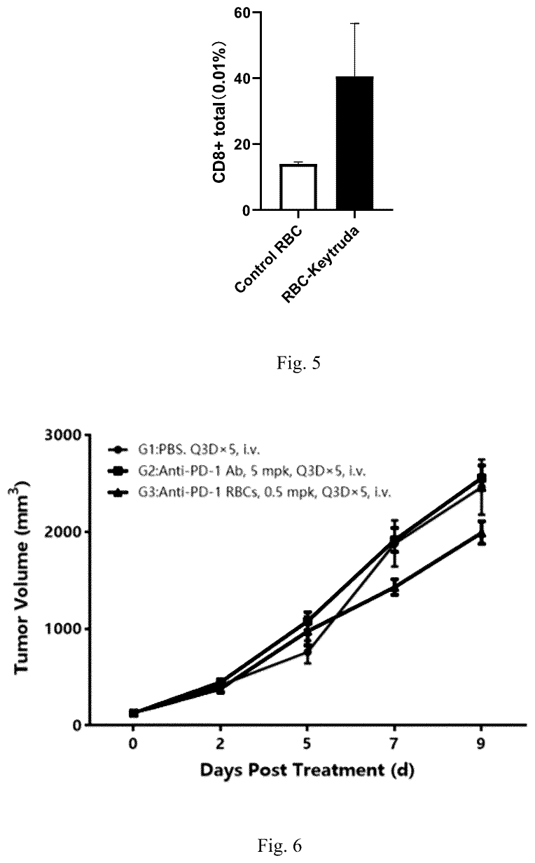

As shown in FIG. 5, after treatment with Keytruda-conjugated mouse engineered red blood cells, on the 39^{th} day following the injection of KP tumor cells, the total number of tumor-infiltrating CD8+ cells in the mice of the RBC-Keytruda treatment group was 2.90 times that of the control group, indicating that RBC-Keytruda can effectively activate the immune system.

Therefore, the Keytruda-conjugated engineered red blood cells exhibited significant tumor-suppressive effects on PD-1 humanized mice harboring the KP tumor cell line.

### Example 5 Treatment of the PB3 Model with SK-RBC

### 1. Research Objective:

The objective of this research was to evaluate the tumoricidal efficacy of Keytruda-conjugated mouse engineered red blood cells in PD-1 humanized mice model harboring the PB3 cell tumor.

### 2. Research Methods

The mouse breast cancer PB3 cells (Epithelial-to-Mesenchymal Transition Contributes to Immunosuppression in Breast Carcinomas, Anushka Dongre, Cancer Res. 2017 Aug. 1; 77 (15): 3982-3989. doi: 10.1158/0008-5472.CAN-16-3292. Epub 2017 Apr. 20.) were subcutaneously injected into a mouse with the cell number of 5×10⁴. In this mouse model, all experimental animals were divided into three groups:
(1) a control group, with blank Control PBS;
(2) a Keytruda monotherapy group; and
(3) a RBCs-Keytruda treatment group.

When the tumor size in the mice reached approximately 100 mm³, the animals were randomly divided into groups with initiation of medication.

The tumor volume was measured with a caliper every 3 days, and all mice were euthanized after administering the treatment four times. The tumor volumes were compared between the two groups of mice.

### 3. Materials and Methods

### 3.1 Preparation of Keytruda-Conjugated Mouse Engineered Red Blood Cells

As described in Example 3, the Keytruda-conjugated mouse engineered red blood cells were prepared, with the difference being that the prepared blood samples were aliquoted according to a 1:5 concentration gradient.
1) 500 μL of RBCs-Keytruda was taken, supplemented with PBS to 1.25 mL, and labeled as “RBCs-Keytruda”.
2) 1.25 mL of PBS was taken labeled as “Control PBS”.
3) To 1213 μL of PBS was added 47 μL of Keytruda, and labeled as “Keytruda”.

### 3.2 Experimental Methods

### Experimental Animals

The animals used in this experiment were PD-1 humanized female C57BL/6 mice, purchased from GemPharmatech. The initial experimental age for all mice was 6 to 8 weeks old.

### Animal Grouping and Administration Methods

### 3.2.1 Animal Administration Methods

Administration route and Administration by intravenous injections via the tail vein method:
Rationale for the The transfusion method for the engineered red blood cell of products was based on the transfusion method for concentrated selection administration route: red blood cell products as stipulated in the “Guidelines on the Quality Monitoring of Whole Blood and Blood Components”.
Administration frequency Once every three days for a total of four injections (q3d×4) and duration:
Administration time: 16:00˜17:00
Administration volume: 200 μL
Administration rate: 25 μL/s, using a syringe to control the rate of administration.

### 3.2.2 Experimental Animal Grouping

| | Number of | Mode of | Administration |
|---|---|---|---|
| Groups | Animals | Administration | Frequency |
| G1: Control | 6 | Intravenous injections | q3 d |
| group, RBC | | via the tail vein | |
| G2: Keytruda | 6 | Intravenous injections | q3 d |
| monotherapy group | | via the tail vein | |
| G3: RBCs-Keytruda | 6 | Intravenous injections | q3d |
| treatment group | | via the tail vein | |

Wherein the PB3 cell was selected as the tumor cell line, which facilitated subcutaneous tumors formation in C57BL/6 mice.

### 4. Experimental Results

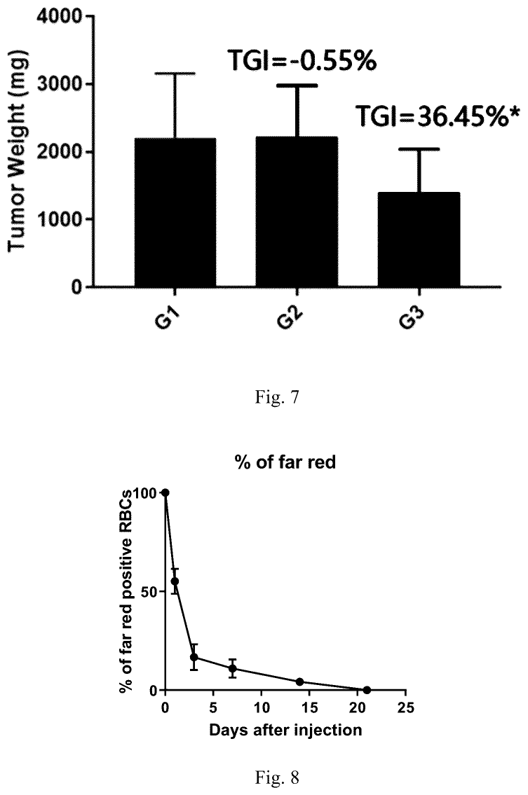

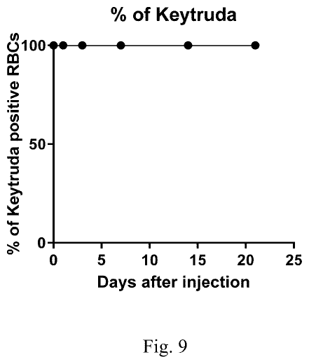

As shown in FIG. 6 (tumor volume change curve) and FIG. 7 (key tumor weight), the tumor sizes in the mice treated with Keytruda-conjugated mouse engineered red blood cells were measured at the endpoint of treatment of the PB3 model, with the RBCs-Keytruda treatment group exhibiting a notable anti-tumor effect (TGItw=36.46%), and displaying a significant statistical difference (*P<0.01), whereas the Keytruda monotherapy group showed no significant anti-tumor effect (TGItw=−0.55%).

### 5. Experimental Conclusions

The Keytruda-conjugated mouse engineered red blood cells treatment group showed a significanttumor-suppressive effect on mice harboring the PB3 mouse breast cancer cell line.

### Example 6 Pharmacokinetics of RBC-Keytruda

### I. Engineered Red Blood Cells can Reach the Spleen

Immunohistochemical staining experiments were conducted to confirm that RBC-Keytruda can reach the spleen. The experimental procedure was as follows:
1. PD-1 humanized mice (purchased from GemPharmatech) aged 6-8 weeks were used, 1×10⁶ mouse lung cancer cell line KP (Shanghai Cell Bank) were subcutaneously injected to establish tumors;
2. When the tumor reached a volume of 100 mm³, two mice were intravenously injected via the tail vein with 200 μL of mouse control red blood cells (RBCs-Control) and 200 μL of engineered red blood cells prepared from Example 1 (conjugated with Keytruda (RBCs-Keytruda)), both of which were labeled with Far-red dye (purchased from Thermo, CellTrace™);
3. Twenty-four hours post-administration, the mice were euthanized, and the spleen tissues were harvested, then cryosection was performed;
4. Immunofluorescence staining was performed on spleen sections from two mice using the Fc antibody (Thermo, Cat. No. 12-4998-82);
5. Images were collected on a confocal microscope, with the Far-red in the far-infrared channel (red) and the Keytruda in the Fc staining channel (green). The experimental results, as shown in FIG. 1A, indicate that the RBCs-Keytruda herein can reach the spleen.

### II. Pharmacokinetics of RBC-Keytruda

### 1. Research Objective:

The objective of this research was to evaluate the pharmacokinetic profile of Keytruda-conjugated engineered red blood cells in mice.

### 2. Research Methods:

The prepared Keytruda-conjugated mouse engineered red blood cells were stained with the cell dye Cell Trace Far Red and administered to C57/BL6 mice via tail vein injection at a dosage of 1×10⁹ cells. In this experiment, the treated engineered red blood cells were injected into three mice, and the injection time was recorded as Day 0. On Days 1, 3, 7, 14, and 21, peripheral blood was collected from the mice, and the proportion of Cell Trace Far Red positive cells in the mouse peripheral blood and the proportion of Keytruda positive cells within the Cell Trace Far Red positive cells were analyzed using flow cytometry to assess the retention of engineered red blood cells in the body and the retention of Keytruda on the engineered red blood cells at different time points.

### 3. Materials and Methods

### 3.1 Preparation of Keytruda-Conjugated Mouse Engineered Red Blood Cells

The Keytruda-conjugated mouse engineered red blood cells RBCs-Keytruda were prepared as described in Example 3. 416 μL of RBCs-Keytruda was taken from the prepared blood samples and supplemented with PBS to a final volume of 1.25 mL, labeled as “RBCs-Keytruda”.

The RBCs-Keytruda, after being supplemented with PBS as described above, was stained with Cell Trace Far Red (Thermo). The specific steps were as follows:
The RBCs-Keytruda cells were centrifuged at 1000×g for three minutes, the supernatant was removed, and resuspended in PBS solution;
Cell Trace Far Red dye was added at a ratio of 1:1000 and the cells were gently resuspended;
the cells were incubated at 37° C. in the dark for 20 minutes;
PBS containing serum was added to the reaction system to stop the reaction, followed by incubation at 37° C. in the dark for 5 minutes;
the cells were centrifuged at 1000×g for three minutes, the supernatant was removed, followed by resuspending in PBS solution for future use.

### 3.2 Experimental Methods

### Experimental Animals

The animals used in this experiment were C57BL/6 mice, purchased from Experimental Animal Center of Westlake University. The initial experimental age for all mice was 6 to 8 weeks old.

### 3.2.1 Animal Administration Methods

Administration route and Administration by intravenous injections via the tail vein method:
Rationale for the The transfusion method for the engineered red blood cell selection of products was based on the transfusion method for concentrated administration route: red blood cell products as stipulated in the “Guidelines on the Quality Monitoring of Whole Blood and Blood Components”.
Administration time: 16:00˜17:00
Administration volume: 200 μL
Administration rate: 25 μL/s, using a syringe to control the rate of administration.

### 4. Experimental Results:

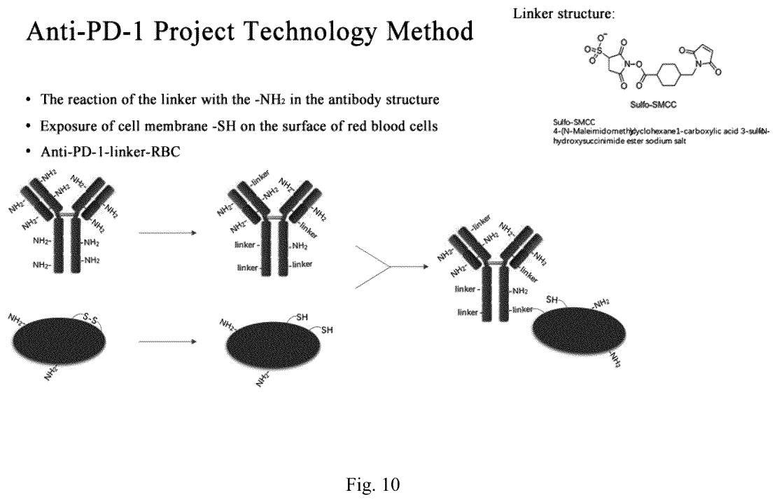

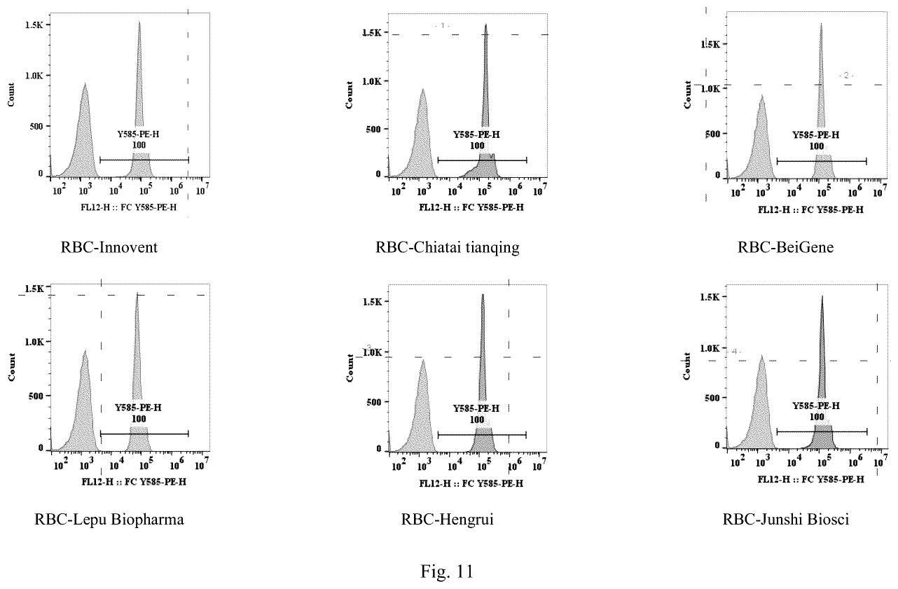

During the pharmacokinetic process, the proportion of Cell Trace Far Red positive cells in the mouse peripheral blood (FIG. 8) and the proportion of Keytruda positive cells within the Cell Trace Far Red positive cells (FIG. 9) were analyzed using flow cytometry. The proportion of the Keytruda-conjugated mouse engineered red blood cells in mice quickly decreased from D0 to D3, then slowly declined from D3 to D21 until the red blood cells were completely cleared. Analysis of the retention of Keytruda on the engineered red blood cells indicated that all Cell Trace Far Red positive cells showed positivity for Keytruda from D0 to D21.

### 5. Experimental Conclusions:

The Keytruda-conjugated engineered red blood cells can remain in the mouse body for up to 21 days and are eventually cleared along with the mouse engineered red blood cells.

### Example 7 Comparisons on the Drug Loading Amount and Relative Activity of Anti-PD-1-RBC Preparations Prepared with Anti-PD-1 Antibodies from Different Brands

### 1. Experimental Objective

The drug loading amount and relative activity of the anti-PD-1-RBC preparations prepared using the process method herein from PD-1 antibodies of different brands was compared.

### 2. Experimental Methods

2.1 Preparations of Anti-PD-1-RBC Antibodies from Different Brands

Antibody brand names (Abbreviations): Hengrui (HR), BeiGene (BJ), Innovent (XD), Merck (K)

According to Example 1, SMCC-conjugated anti-PD-1 antibodies from different brands, namely SMCC-HR, SMCC-BJ, SMCC-XD, and SMCC-K, as well as anti-PD-1-RBC preparations of human blood RBCs conjugated with anti-PD-1 antibodies from different brands (K-hRBC-211227, BJ-hRBC-211227, HR-hRBC-211227, and XD-hRBC-211227) were prepared.

According to Example 3, the anti-PD-1-RBC preparations of mouse blood RBCs conjugated with anti-PD-1 antibodies from different brands, namely K-mRBC-211227, BJ-mRBC-211227, HR-mRBC-211227, and XD-mRBC-211227 were prepared.

### 2.2 Methods for In Vitro Relative Activity Testing of Antibodies and Preparations

Refer to the test methods described in Example 2.

### 2.3 Determination of Drug Loading Amount in Anti-PD-1-RBC Preparations

### 2.3.1 Experimental Procedure

In this experiment, an ELISA method was employed to detect the content of anti-PD-1 antibodies in the anti-PD-1-RBC preparation prepared in step 1. The specific procedure was as follows:

### 2.3.1.1 Preparation of Solutions

Antigen solution preparation: 1 mL of sterile water was added to the PD-1 antigen lyophilized powder (ACRO, PD1-H522) to dissolve the powder completely to a concentration of 25 mg/mL. The solution was aliquoted and stored at a temperature between −80° C. and −60° C., with a shelf life of 3 months.

Blocking solution: 2% BSA (Solarbio, A8020)−PBS, 2.0 g of BSA was weighed and added to 100 mL of PBS solution until completely dissolved, and the solution was stored at a temperature between 2° C. and 8° C., with a shelf life of 7 days.

Washing solution: PBST, 1×PBS was mixed with 0.05% Tween 20 (Sangon, A600560-0500) and store at room temperature, with a shelf life of 30 days.

Sample dilution fluid: 0.1% BSA-PBST Solution, 0.1 g of BSA was weighed and added to 100 mL of 0.1% PBST solution until completely dissolved, or 1 mL of 2% BSA-PBS was taken and added to 19 mL of 0.1% PBST, followed by mixing thoroughly and storing at a temperature between 2° C. and 8° C., with a shelf life of 7 days.

### 2.3.1.2 ELISA Test Procedure

Coating: the aforementioned prepared human PD-1 antigen (PD1 Leu25-Gln167 (Accession #NP_005009.2)) solution was diluted to a concentration of 500 ng/ml with PBS, and coated on the ELISA plate, the plate was incubated overnight at a temperature between 2° C. and 8° C.

Washing plates: the plate was washed 3 times with the washing solution.

Blocking: the blocking solution was added to the 96-well plate and incubated at 37° C. for 1-2 hours.

Sample addition: 50 μL of the anti-PD-1-RBC preparation diluted by 16,000 and 32,000 times, as well as various brands of anti-PD-1 antibodies diluted linearly (0-2 μg/mL) were taken and added to the 96-well plate, with two parallel wells for each, and the plate was incubated at 37° C. for 1-2 hours.

Washing plates: the plate was washed 4 times with the washing solution.

Addition of secondary antibody: the secondary antibody (Abcam, ab97225) was diluted with PBS, then added to the 96-well plate at 100 μL per well.

Color development: 100 μL/well of tetramethylbenzidine (TMB, SURMODICS, TMD-1000-01) solution in methanol was added, and the plate was incubated in the dark at 37° C. for 15-20 minutes to allow color development.

Termination: 50 μL/well of the stop solution (Solarbio, CL058) was added.

Detection and reading: the optical density absorbance values were read at OD 450 nm-570 nm using a microplate reader.

### 2.3.2 Result Calculation

data processing software was utilized to calculate and analyze the data. The mean OD values and % CV for both the samples to be tested and the standards in duplicate wells were calculated. A standard curve was plotted employing a four-parameter logistic regression model with the concentrations of the standards as the X-axis and the mean OD values at 450-570 nm as the Y-axis, and the R² of the fitted curve was calculated (the formula is provided below).

$$y = D + ( A - D ) / ( 1 + ( X / C ) \hat{} B )$$

The concentration of PD-1 antibodies (μg/mL) in both standards and samples to be tested was calculated using the mean OD values from duplicate wells by the fitted curve.

### 3. Experimental Results

3.1 Determination of Relative Activity of SMCC Reacted with Antibodies from Different Brands

| | Relative activity (with |
|---|---|
| Lot No. | this antibody as control) |
| SMCC-K | 91% |
| SMCC-BJ | 83% |
| SMCC-HR | 91% |
| SMCC-XD | 107% |

In vitro testing of the relative activity of the reaction products after the reaction of Sulfo-SMCC with PD-1 antibodies from different brands was conducted. The results indicated that under the current process conditions, the reaction itself of Sulfo-SMCC with the antibody has a minimal impact on the activity of the antibody.

3.2 Determination of Drug Loading Amount and Relative Activity of Human Blood Preparations Prepared from Antibodies of Different Brands

| Lot No. of human | Drug loading |
|---|---|
| blood preparation | amount (μg/mL) |
| K-hRBC-211227 | 109.15 |
| BJ-hRBC-211227 | 116.47 |
| HR-hRBC-211227 | 85.71 |
| XD-hRBC-211227 | 52.24 |

The engineered red blood cells loading PD-1 antibodies can be prepared using human blood preparations prepared from different brands of PD-1 antibodies, with a drug loading amount ranging from 52.24 to 109.15 μg/mL.

3.3 Determination of Drug Loading Amount of Mouse Blood Preparations Prepared from Antibodies of Different Brands

| Lot No. of mouse | Drug loading |
|---|---|
| blood preparation | amount (μg/mL) |
| K-mRBC-211227 | 274.33 |
| BJ-mRBC-211227 | 140.95 |
| HR-mRBC-211227 | 194.71 |
| XD-mRBC-211227 | 50.84 |

The engineered red blood cells loading PD-1 antibodies can be prepared using C57bl/6 mouse blood preparations prepared from different brands of PD-1 antibodies, with a drug loading amount ranging from 50.84 to 274.33 μg/mL.

### Example 8 Comparison of the Drug Loading Amount and Relative Activity of Anti-PD-1-RBC Preparations Prepared from Anti-PD-1 Antibodies of Different Brands

### 1. Experimental Objective

The coupling efficiency, in vitro activity, and drug loading amount of anti-PD-1-RBC preparations prepared with PD-1 antibodies from different brands using the process methods herein were compared.

### 2. Experimental Methods

2.1 Preparation of Anti-PD-1-RBCs with Antibodies from Different Brands and their Coupling Efficiencies

In reference to Example 3, anti-PD-1 antibodies from different brands conjugated with SMCC, as well as anti-PD-1-RBC preparations with mouse blood RBCs conjugated to different brands of anti-PD-1 antibodies were prepared. The specific brands and the designations of the anti-PD-1-RBC preparations are presented in the table below:

Antibody brands and sources

| Sources of PD-1 antibody brands | Anti-PD-1-RBC preparations |
|---|---|
| PD-1 antibody from Innovent | RBC-Innovent |
| PD-1 antibody from Chiatai tianqing | RBC-Chiatai tianqing |
| PD-1 antibody from BeiGene | RBC-BeiGene |
| PD-1 antibody from Lepu Biopharma | RBC-Lepu Biopharma |
| PD-1 antibody from Hengrui | RBC-Hengrui |
| PD-1 antibody from Junshi Biosci | RBC-Junshi Biosci |
| Nivolumab PD-1 antibody | RBC-Nivolumab |
| (Nivolumab from BMS) | |
| PD-1 antibody from Henlius Biotech | RBC-Henlius Biotech |
| PD-1 antibody from Gloria Biosci | RBC-Gloria Biosci |

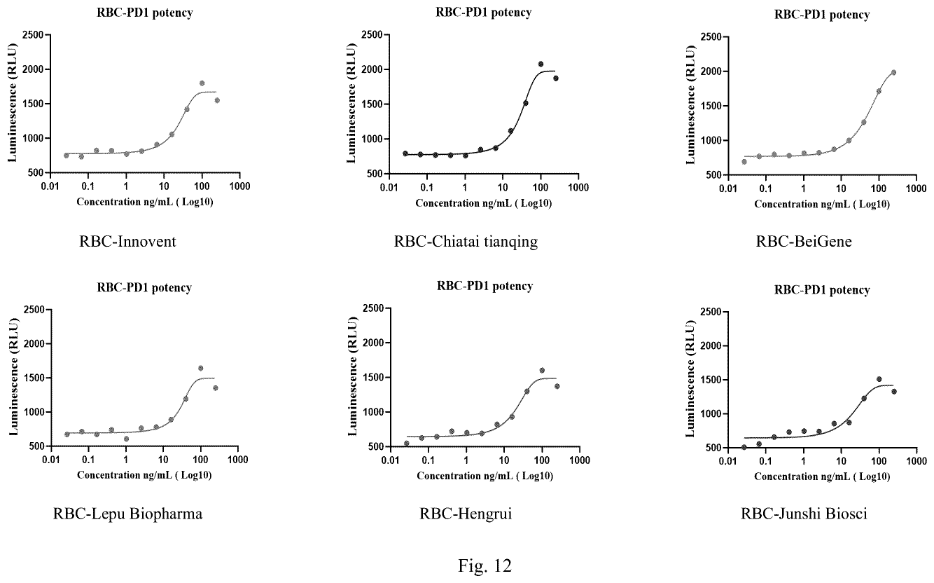

The antibody coupling efficiency was assayed similarly to Example 3-2.1, wherein the sample was labeled with the flow cytometry antibody Goat anti-Human IgG Fc (PE anti-Human IgG Fc Secondary Antibody) at a dilution of 1:200 for 2 minutes, and subjected to flow cytometry assay using the PE channel, to test the coupling efficiency of PD-1 antibodies from various brands with red blood cells, with the results showing the coupling efficiency of each brand of approximately 100% (FIG. 11).

Samples were labeled with FC flow antibody 1:200 for 2 minutes, followed by flow cytometry assay.

### 2.2 In Vitro Activity Assay Method for Preparations

As described in aforementioned Example 2, the PD-1 expressed in Jurkat-PD-1 Effector Cells can bind to the ligand PD-L1 on PD-L1 aAPC/CHO-K1 Cells, thus inhibiting the generation of signals. The binding of PD-1 proteins of the red blood cell preparations from various brands to PD-L1 can block the binding of PD-L1 and PD-1, thereby activating the downstream pathways to produce signals.

The in vitro relative activity was determined by referencing the test method described in Example 2, wherein the starting highest concentration of red blood cell preparations from various brands of antibodies applied was 250 ng/mL, with a gradient dilution of 2.5×.

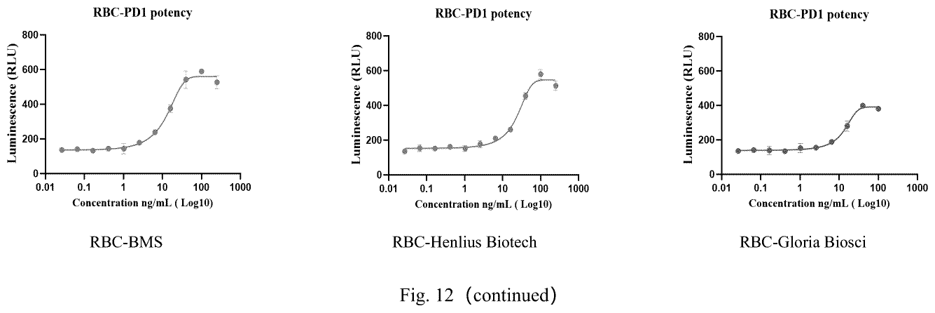

The results were shown in FIG. 12, which indicate that the red blood cell conjugated with PD-1 antibody preparations (anti-PD-1-RBC preparations) exhibit significant in vitro activity.

### 2.3 Pharmacokinetics of Anti-PD-1-RBC Preparations

Similar to the section “II. Pharmacokinetics of RBC-Keytruda” as described in Example 6, the report evaluating the pharmacokinetics of mouse engineered red blood cells conjugated with antibodies from various brands in mice, as well as the preparations, materials, and injection techniques for the red blood cell preparations of each brand were referred to in Example 6-II-3.

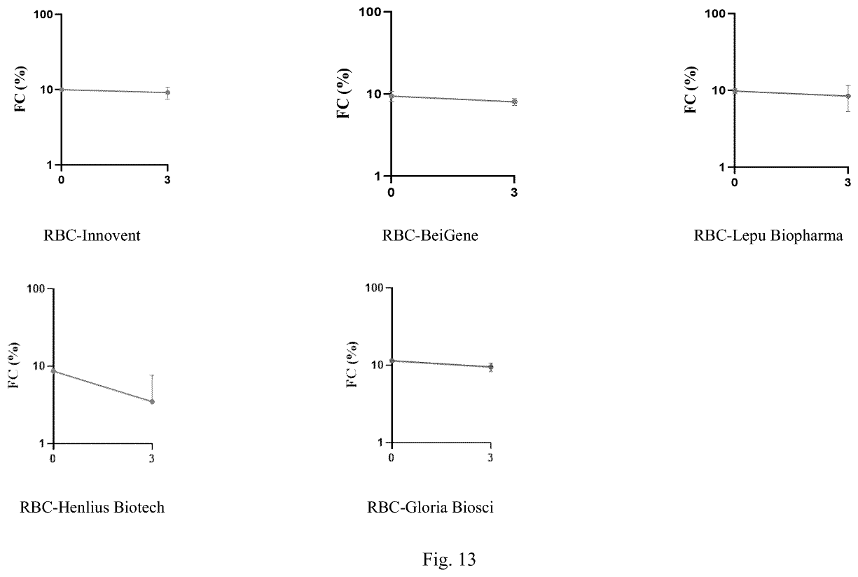

By flow cytometry assay, the Fc region of antibodies in mouse peripheral blood was detected at 30 minutes post-injection (designated as 0) and on D3 (the third day, designated as 3) using the flow cytometry antibody Goat anti-Human IgG Fc, with the theoretical value of 10%. The results were shown in FIG. 13, indicating that the red blood cell-conjugated PD-1 antibody preparations can stably exist in mice.

### 2.4 Determination of Drug Loading Amount of Anti-PD-1-RBC Preparations

The content (the drug loading amount of preparation) of anti-PD-1 antibodies in the prepared anti-PD-1-RBC preparations was determined using the ELISA method as described in Example 7-2.3. The results are presented in the table below:

| Identification | | Drug loading amount |
|---|---|---|
| Number | Preparations | (μg/10¹⁰ cells) |
| 1 | RBC-Innovent | 62.36 |
| 2 | RBC-Chiatai tianqing | 102.63 |
| 3 | RBC-BeiGene | 152.42 |
| 4 | RBC-Lepu Biopharma | 53.92 |
| 5 | RBC-Hengrui | 130.00 |
| 6 | RBC-Junshi Biosci | 116.28 |
| 7 | RBC-Nivolumab | 162.7 |
| 8 | RBC-Henlius Biotech | 138.9 |
| 9 | RBC-Gloria Biosci | 27.3 |

The engineered red blood cells carried with PD-1 antibodies can all be prepared with human blood preparations prepared with PD-1 antibodies from different brands, with a drug loading amount ranging from 27.3 to 162.7 μg/10¹⁰ cells.

What is claimed is:

1. An antibody-erythrocyte conjugate, comprising an erythrocyte conjugated with an antibody or antigen-binding fragment thereof,
wherein the antibody or antigen-binding fragment thereof is conjugated to a membrane protein on the erythrocyte via a linker,
the antibody or antigen-binding fragment thereof is a PD-1 antibody or antigen-binding fragment thereof, and the erythrocytes are mature natural erythrocytes.

2. The antibody-erythrocyte conjugate of claim 1, wherein the antibody or antigen-binding fragment thereof comprises CDR1, CDR2, and CDR3 of the variable region of a heavy chain, as well as CDR1, CDR2, and CDR3 of the variable region of a light chain, wherein the CDR1, CDR2, and CDR3 of the variable region of a heavy chain and the CDR1, CDR2, and CDR3 of the variable region of a light chain are derived from the antibodies disclosed in CN108473977B, CN104250302B, CN105531288B, CN105026428B, WO2008156712A1, or WO2006121168A1, or pembrolizumab, camrelizumab, tislelizumab, sintilimab, toripalimab, zimberelimab from Gloria Biosci, penpulimab from Chiatai tianqing, pucotenlimab from Lepu Biopharma, serplulimab from Henlius Biotech, or nivolumab.

3. The antibody-erythrocyte conjugate of claim 1, wherein the antibody or antigen-binding fragment thereof is selected from the antibodies disclosed in CN108473977B, CN104250302B, CN105531288B, CN105026428B, WO2008156712A1, or WO2006121168A1, or selected from pembrolizumab, camrelizumab, tislelizumab, sintilimab, toripalimab, zimberelimab from Gloria Biosci, penpulimab from Chiatai tianqing, pucotenlimab from Lepu Biopharma, serplulimab from Henlius Biotech, or nivolumab or antigen-binding fragments thereof.

4. The antibody-erythrocyte conjugate of any of claims 1-3, wherein the antigen-binding fragment is selected from a Fab, a Fab′, a Fab′-SH, a Fv, a single-chain antibody (e.g., scFv), a (Fab′)2, a single-domain antibody such as VHH, a dAbs (domain antibody), or a linear antibody or a half-antibody.

5. The antibody-erythrocyte conjugate of any of claims 1-3, wherein the linker has functional groups that can react with the thiol groups on cysteine residues present on the membrane proteins of RBCs to form a covalent bond to attach to RBCs, and has functional groups that can conjugate to the antibody by reacting with the amino groups (—NH₂) on lysine in the antibody structure.

6. The antibody-erythrocyte conjugate of any of claims 1-3, wherein the linker is LC-SMCC, SMCC, or Sulfo-SMCC.

7. The antibody-erythrocyte conjugate of any of claims 1-3, wherein the erythrocytes are obtained by the following methods, the methods comprising:
(i) isolating and concentrating the erythrocytes from whole human blood, and optionally removing the leukocytes by filtration;
(ii) treating the erythrocytes with a thiol-reducing agent to chemically modify the surface of the erythrocytes; and
(iii) collecting and concentrating the modified erythrocytes.

8. The antibody-erythrocyte conjugate of claim 7, wherein the thiol-reducing agent is TCEP.

9. A method for preparing an antibody-erythrocyte conjugate of any of claims 1-8, which comprising:
(1) allowing the nucleophilic groups of the antibody or antigen-binding fragment thereof to react with a divalent linker reagent to form the antibody or antigen-binding fragment thereof connected to the linker via covalent bonds;
(2) chemically modifying the surface of mature natural erythrocytes to expose nucleophilic groups of the membrane proteins on the erythrocytes;
(3) mixing the antibody or antigen-binding fragment thereof connected to the linker from (1) with the erythrocytes obtained from (2), allowing a covalent conjugation of the two via the linker; and
(4) collecting the antibody-erythrocyte conjugates obtained from (3).

10. The method of claim 9, wherein the nucleophilic group on the antibody or antigen-binding fragment thereof is an N-terminal amino group or a side-chain amino group, and the nucleophilic group on the membrane protein of red blood cell is a side-chain thiol group.

11. The method of claim 10, wherein the side-chain thiol group is a thiol group resulting from the reduction of cysteine.

12. The method of claim 10, wherein a thiol-reducing agent is applied for the chemical modification of red blood cells.

13. The method of claim 12, wherein the thiol-reducing agent is TCEP.

14. The method of claim 13, wherein the final concentration of TCEP is between 0.1 mM and 50 mM, for example, between 0.5 mM and 10.0 mM, 0.5 mM and 5.0 mM, preferably approximately 5.0 mM.

15. A pharmaceutical composition or preparation, comprising the antibody-erythrocyte conjugate of any of claims 1-8, or the antibody-erythrocyte conjugate prepared by the method of any of claims 9-14, and pharmaceutical excipients.

16. The pharmaceutical composition or preparation of claim 15, wherein the preparation is a human blood preparation, such as autologous or allogeneic human blood preparation.

17. The pharmaceutical composition or preparation of claim 16, wherein the human blood preparation is a human leukoreduced blood preparation.

18. The pharmaceutical composition or preparation of claim 16 or 17, wherein the human blood preparation comprises an antibody-erythrocyte conjugate at a concentration of 10-1000 μg/mL, for example, the antibody-erythrocyte conjugate at concentrations of 50 μg/mL, 60 μg/mL, 70 μg/mL, 80 μg/mL, 90 μg/mL, 100 μg/mL, 150 μg/mL, 200 μg/mL, 250 μg/mL, 300 μg/mL, 350 μg/mL, 400 μg/mL, 450 μg/mL, or 500 μg/mL or more.

19. A use of the antibody-erythrocyte conjugate of any of claims 1-8, or the antibody-erythrocyte conjugate prepared by the methods of any of claims 9-14 in the preparation of pharmaceuticals or human blood preparations, wherein the pharmaceuticals or human blood preparations are used to stimulate the immune system of (for example, to enhance the immune response of cells) of the host or to treat cancer in a subject.

20. The use of claim 19, wherein the cancer is the one that is resistant or insensitive to anti-PD-1 antibodies.

21. The use of claim 19, wherein the cancer is the one characterized by elevated protein levels and/or nucleic acid levels (e.g., increased expression) of PD-1, PD-L1, and/or PD-L2 in tumor cells, such as compared to the normal cells in corresponding tissues of healthy individuals, or compared to the protein levels and/or nucleic acid levels of PD-1, PD-L1, and/or PD-L2 in normal cells of healthy tissue adjacent to tumor tissue.

22. Use of any of claims 19-21, wherein the cancer is a gastrointestinal tumor, such as colon cancer or colorectal cancer, pancreatic cancer, lung cancer, or breast cancer.

23. Use of any of claims 19-22, wherein the red blood cells in the antibody-erythrocyte conjugate are derived from the subject to be treated or a healthy subject.

24. Use of any of claims 19-23, wherein the pharmaceutical or human blood preparation is co-administered with one or more therapeutic methods or other therapeutic agents.

25. The use of claim 24, wherein the therapeutic method is radiotherapy or surgical treatment, and/or the therapeutic agents are selected from chemotherapeutic agents, other antibodies, cytotoxic agents, vaccines, etc., preferably, the therapeutic agent is selected from tumor vaccines, immune checkpoint inhibitory antibodies, or immune agonist antibodies.
